(19) United States
(12) Patent Application Publication
(10) Pub. No.: US 2006/0127367 A1
(43) Pub. Date: Jun. 15, 2006

(54) Compositions containing macrophages and uses thereof

(76) Inventors: Benedicte Chazaud, Paris (FR); Romain Gherardi, Brunoy (FR); Luc Hittinger, Boissy Saint Leger (FR); Emmanuel Teiger, Paris (FR)

Correspondence Address: YOUNG & THOMPSON, 745 SOUTH 23RD STREET, 2ND FLOOR, ARLINGTON, VA, 22202, US

(21) Appl. No.: 10/521,738

(22) PCT Filed: Jul. 15, 2004
(86) PCT No.: PCT/EP04/07891

§ 371 Date: Nov. 1, 2005

(30) Foreign Application Priority Data
Jul. 16, 2003 (WO) PCT/EP03/07731

(51) Int. Cl. A61K 35/14

(52) U.S. Cl. 424/93.7

(57) ABSTRACT

The invention relates to the use of macrophages for the preparation of a drug for the treatment of a disease or of a lesion involving cellular apoptosis, reduction of the survival of cells and/or destruction of cells.

The present invention relates to a new use of macrophages and to new compositions containing them, in particular for the treatment of a disease or a lesion involving either cellular apoptosis, reduction of the survival of cells and/or destruction of cells. It also relates to their use for the preparation of a drug for improving the survival of precursor cells or stem cells. It also relates to pharmaceutical compositions containing macrophages and progenitors cells or stem cells, and their use for treating a disease or a lesion involving cellular destruction.

Stem cells or precursor cells may be used for engrafting a mammal suffering from a disease or a lesion in which is involved some cellular destruction. However, engraftment of precursor cells or stem cells for tissue repair is restricted by the fact that an important proportion of the engrafted cells die, even in the absence of an immune response against the graft, when autologous cells are administered. Furthermore, the post lesional reconstitution of tissues with adequate structure and functionality is difficult to obtain.

Adult skeletal muscle regeneration results from activation, proliferation and fusion of myogenic precursor cells (mpc) residing beneath muscle fiber basal lamina, the so-called satellite cells {Hawke & Garry 2001 204/id}. Myogenic precursor cells are capable of proliferating and of fusing to repair or replace a damaged muscle fiber.

Numerous attempts of mpc transplantation in skeletal muscle have been performed in both animals and humans. Encouraging results were obtained in experimental conditions hardly applicable to humans, including engraftment of myogenic cell lines or recipient irradiation (Grounds, 1996). Except the controversial results of Law and his group (1997), attempts using primary culture-derived normal mpc injected to untreated dystrophin-deficient muscle (in mdx mice or humans) failed to improve muscle strength (Skuk et Tremblay, 2000). Main limitations to efficient cell therapy in skeletal muscle include two factors:
lack of transplanted cell diffusion in the engrafted muscle, which still precludes efficient restorative cell therapy of diffuse muscle disorders, since mpc injections at hundreds to thousands sites would be unethical (Skuk et Tremblay, 2000).
acute death of transplanted cells, which is observed even in autografts or immunosupressed recipients. It consists of massive cell mortality occurring within 24-48 hours post-injection. This poorly understood phenomenon is distinct from rejection (Skuk et Tremblay, 2000). It is reminiscent of caspase-dependent apoptosis of embryonic neurons engrafted into striatum of mice or patients with Parkinson disease (Schierle et al, 1999).

Transfection of engrafted mpc by the interleukin (IL)-1 antagonist IL-1-Ra was able to prevent their acute death (Qu et al, 1998). This result is consistent with IL-1β-induced mpc apoptosis (Authier et al, 1999). Mpc acute death may be decreased by transfection with the TGF β-1 gene (Merly et al, 1998) or administration of anti-LFA-1 (αL β2 integrin) antibodies. The latter effect was not observed with anti-mac-1 antibodies, suggesting a crucial role of neutrophil degranulation, rather than modulation of macrophage function, in mpc acute death (Guérette et al, 1997). Acute mpc deprivation in survival cues likely participates to massive death of mpc (Grounds, 1996). Indeed, during regeneration, angiogenesis is also essential for muscle regeneration. Similar observations made in other systems built up the concept of supportive stroma encompassing all microenvironmental cues influencing the fate of adult stem cells, i.e. controlling quiescence, self-renewal, proliferation and differentiation (Spradling et al, 2001).

Coronary weakness, and its main consequence, myocardial infarction, represent the first cause of hospitalization in cardiology services. In absence of early treatment, myocardial infarction leads to the ischaemic necrosis of the myocardial territory located downstream the artery obstruction by a clot. When it is constituted, necrosis is irreversible, the actual treatments (inhibitors of conversion enzyme, beta-blockers, anti-thrombotics and treatment of risk factors) only avoid secondary complications. The more extended is the necrosis, the more probable is the risk of evolution through cardiac insufficiency or death of the patient.

Muscle cell transplantation in heart was performed in order to replace missing cardiomyocytes by contractile cells, to limit post-infarction akinetic fibrous scar formation and subsequent congestive heart failure. Successful preclinical studies using foetal cardiomyocytes and myogenic cells lines cannot be transferred to humans, due to ethical reasons and poor availability, or to potentially tumorigenic properties of the cells, respectively. It has been shown that autologous mpcs may generate functional tissue (Taylor et al, 1998; Menasché et al, 2001), although mechanisms by which en grafted cells improved myocardial contractility remained elusive. Also, acute and massive death of transplanted cells is the main limitation of mpc transplantation (Ménasché, 2002).

Muscle adult stem cell transfer in skeletal muscle improve graft efficiency as compared to myogenic cells transplantation. This effect was attributed to a better adult muscle cell survival and a better capacity to fuse with host myofibers (Lee, 2000).

Stem cells transplantation has also been attempted for the treatment of Parkinson disease and caused beneficial effects limited in time.

Macrophages are commonly known as phagocytosing immune cells (Meszaros et al, 1999). They also secrete factors such as chemokines or cytokines. In addition to phagocytosis and antigen presentation, these cells may play a supportive role through a varied repertoire of plasma membrane and secreted molecules {Gordon 1995 433/id}, as previously shown for erythroblasts, hepatocytes and neurons {Sadahira & Mori 1999 355/id} {Takeishi, Hirano, et al. 1999 699/id} {Polazzi, Gianni, et al. 2001 701/id}. These are normal physiologic conditions, and not post-lesional tissue repair. The phagocytic potential of muscle resident macrophages is debated, but it is generally accepted that newly recruited macrophages actively remove necrotic debris to facilitate subsequent muscle regeneration {McLennan 1996 153/id} {Pimorady-Esfahani, Grounds, et al. 1997 278/id}. Furhtermore, through the factors secreted, macrophages have been a recognized pro-angiogenic capacity.

The present invention provides the use of macrophages for the preparation of a drug for the treatment of a disease or of a lesion involving cellular apoptosis, reduction of the survival of cells and/or destruction of cells.

The present invention also provides the use of macrophages for the preparation of a drug for the improvement of survival of a first type of cells, for the treatment of a disease or of a lesion involving the destruction of a second type of cells or of a tissue containing a second type of cells, said first type of cells being chosen among the group consisting of: precursor cells and stem cells, said second type of cells being chosen among the group consisting of: precursor cells, stem cells and any type of differentiated cells.

The inventors surprisingly showed that macrophages may inhibit precursor cells apoptosis in a cell to cell contact and may serve as stromal support for efficient cellular engraftment for tissue repair. They showed in particular that macrophages could inhibit myogenic precursor cells apoptosis.

By “macrophages” is meant cells exhibiting properties usually described for macrophages, including phagocytosis, expression of defined cell surface markers such as CD64, CD14 and HLA-DR antigen expression. Macrophages according to the invention can be isolated from tissues or preferentially by ex vivo differentiation from blood monocytes, bone marrow precursor cells or from any other possible precursor, and by using any differentiation method, precursors and methods being known by any person skilled in the art.

By “precursor cells” is meant non terminally differentiated tissue cells, still having a proliferative capacity. By “stem cell” is meant adult stem cell, excluding embryonic stem cells. Precursor and stem cells according to the invention may originate from different tissues peripheral blood, bone marrow, haematopofetic cells, mesenchymal tissue, muscle, fat tissue.

By “mammal” is meant any mammal including humans.

In a particular embodiment of the invention, said first type of cells is to be grafted into a mammal for the treatment of one or several focal lesions or dysfunction. The presence of focal lesions allows the engraftment of the animal, which would be very difficult to reduce to practice and unethical for treating many disseminated lesions.

In an other particular embodiment, said first type of cells and/or said macrophages are autologous for said mammal. Grafted cells or tissues may be heterologous to the mammal, but for limiting the possibility of immune reactions between grafted cells and hosts, the use of autologous cells are preferable.

In another particular embodiment, said lesion is a bone or muscular lesion, possibly resulting from a disease or an injury. It can be for example a bone fracture, a torn muscle, or a destruction of a tissue containing said second type of cells, which can be differentiated cells, precursor or stem cells. In a particular embodiment, said pathology is a tumor-associated disease, which may have necessitated surgery for ablating tumoral cells leading to the destruction of environment tissues.

In a more particular embodiment of the invention, said lesion is a cardiac lesion or injury. In particular, it can be for example myocardial infarction, heart insufficiency, coronary thrombosis, dilated cardiomyopathy or any cardiomyocyte dysfunction subsequent to, or resulting from, any genetic defect. For example, the invention could be useful in case of acute cardiac insufficiency, with patients needing circulatory assistance, to reduce the duration of said assistance. The invention could also be used in case of cardiac insufficiency with bad prognostic despite progress in treatments, such as infiltrative cardiomyopathy, or cardiomyopathy due to anthracyclins toxicity or cardiomyopathy secondary to VIH infection (Felker, N Engl J Med, 2000; 342: 1077).

The present invention also relates to the use of macrophages as inhibitors of apoptosis of precursor or stem cells. When cells suffer from deprivation of factors essential for survival, they enter into an apoptosis process. The inventors have surprisingly found that macrophages could improve the survival of precursor cells and/or stem cells, and in particular that macrophages could, at least partially, lower apoptosis of said precursor cells and/or stem cells. Said lowering of apoptosis appears to be mainly mediated via direct cell to cell contact. Apoptosis level can be assessed for example by determination of oligosomal DNA levels, annexin V labeling or caspase 3 activity measurements, or by any other technique known by a person skilled in the art. The inventors also surprisingly found that the presence of precursor or stem cells could lower the apoptosis affecting macrophages. Each of macrophages and precursor or stem cells could exert a reciprocal effect lowering the apoptosis level of the other type of cells.

In a more particular embodiment, the present invention also relates to the use of macrophages as stromal support for precursor or stem cells. The inventors found that macrophages could act as a stromal support for precursor cells or stem cells, by inhibiting apoptosis, enhancing proliferation of cells and providing favorable environment for cell growth and differentiation, via cytokines and growth factors production. Macrophages could also favor the diffusion of transplanted cells via their angiogenic properties.

Tissue-specific microenvironmental cues delivered by stromal components influence the fate of both adult stem cells and their progeny {Spradling, Drummond-Barbosa, et al. 2001 46/id}. The stem cell niche represses differentiation of quiescent and self-renewing cells whereas the stromal support promotes cell survival and proliferation and appears essential for differentiation of cells escaped from the niche {Spradling, Drummond-Barbosa, et al. 2001 46/id}. Mpc likely depend on such a stromal support to develop their myogenic program {Seale, Asakura, et al. 2001 446/id}. Recruited macrophages act as potent supportive cells for mpc through delivery of soluble mitogenic factors and cell contact-mediated survival signals.

Said precursor or stem cells may come from tissue or from peripheral blood (Sata et al, 2002, Zhao et al, 2003), and may be chosen among a group consisting of: myogenic precursor cells, endothelial precursor cells, hematopoietic precursor cells, bone marrow precursor cells, mesenchymal precursor cells, adipocyte precursor cells, neuronal precursor cells and multipotent adult stem cells.

In a more particular embodiment, the present invention provides a composition containing myogenic precursor cells (mpc).

In a particular embodiment, the present invention provides a composition containing macrophages and precursor or stem cells from muscle, from bone marrow, peripheral blood or from any other tissue.

The present invention also provides the use of a pharmaceutical composition containing macrophages and at least one first type of cells, in association with a pharmaceutically acceptable vehicle, for the preparation of a composition to be grafted into a mammal, said first type of cells being chosen among the group consisting of: precursors cells and stem cells.

Said composition contains only clinical grade products for administration to human beings. Any vehicle, carrier, auxiliary agent and formulation adopted in art for manufacturing compositions to be administered into a mammal, and particularly into a human being, can be used in the composition according to the invention. A skilled person can identify said components and all the steps of the relevant process of manufacturing.

In a particular embodiment, said composition contains precursor or stem cells and/or macrophages autologous to the mammal to be grafted. For the reasons cited before, autologous precursor or stem cells and macrophages are preferred.

In another particular embodiment, a composition according to the invention is used for the treatment of a disease or a lesion involving the destruction of cells. The present invention is useful in the case of diseases, wounding or injuries resulting in the destruction of cells and/or at least parts of tissues, which may lead to loss of functionality.

In a particular embodiment, said disease or injury results in only some focal lesions, rather than many disseminated lesions.

In a more particular embodiment, destruction of cells or of at least parts of a tissue may result from surgical intervention intended to remove non-functional or tumoral cells or tissues. Said destruction of cells or tissues may occur in bones, muscles or any other organ. In a more particular embodiment, the use of a composition according to the invention takes place for the treatment of heart muscle diseases, said cardiac lesion being possibly myocardial infarction, coronary thrombosis, dilated cardiomyopathy or any cardiomyocyte dysfunction subsequent to, or resulting from, any genetic defect.

In a more particular embodiment, compositions used according to the invention contain macrophages and myogenic precursor cells. It has been shown that compositions containing myogenic precursor cells could be used for graft in skeletal and in cardiac muscles.

In another particular embodiment, compositions according to the invention contain macrophages and precursor or stem cells; when expressed as a percentage of the total number of cells present in the composition, macrophages and precursors or stem cells represent at least about 70%, and preferably about 90% of the total number of cells. Other cells may be fibroblasts or stromal cells. Cells can be identified, characterized and numbered by techniques known by a skilled person, such as Fluorescent Activated Cells Sorting performed on cell populations previously incubated with labeled antibodies specific for cell determinants. As an example, macrophages may be characterized by using anti-CD64 antibodies, mpc with anti-CD56 antibodies and blood stem cells by anti-CD34 antibodies. In a preferred embodiment of the invention, compositions according to the invention contain from about 80 to about 100% of macrophages and precursor or stem cells, and more preferably about 90% of macrophages and precursor or stem cells.

In a more particular embodiment, within the population of cells identified as precursor or stem cells, also called “first type of cells” and as macrophages, the ratio between the number of the first type of cells and the macrophages is comprised between about 1/20 and about 50/1, preferably between about 1/10 and about 10/1, more preferably between about 1/5 and about 5/1, more preferably between about 1/2 and about 2/1, and more preferably of about 1/1, the number of precursor or stem cells and of macrophages being approximately equivalent.

In another particular embodiment, the composition used according to the invention contains from about 0.5 10⁸ to about 7.5 10⁸ macrophages and from about 0.5 10⁸ to about 7.5 10⁸ of said first type of cells.

The present invention also relates to a pharmaceutical composition containing at least one first type of cells, said first type of cells being possibly precursor cells or stem cells, and macrophages, in association with a pharmaceutically acceptable vehicle.

In a particular embodiment, a pharmaceutical composition of the invention contains a first type of cells is chosen among a group consisting of: myogenic precursor cells, endothelial precursor cells, hematopoietic precursor cells, bone marrow precursor cells, mesenchymal precursor cells, neuronal precursor cells and multipotent adult stem cells.

In a more particular embodiment, a pharmaceutical composition of the invention contains a first type of cells and macrophages, wherein the ratio between said first type of cells and macrophages, as expressed in number of cells, is comprised between about 1/20 and about 50/1, preferably between about 1/10 and about 10/1, more preferably between about 1/5 and about 5/1, more preferably between about 1/2 and about 2/1, and more preferably of about 1/1, the number of precursor or stem cells and of macrophages being approximately equivalent.

In another particular embodiment, a pharmaceutical composition of the invention contains a first type of cells and macrophages wherein the ratio between said first type of cells and macrophages, as expressed in number of cells, is comprised between about 1/10 and about 10/1, and is preferably of about 1/1.

In another particular embodiment, a pharmaceutical composition according to the invention contains stem cells or precursor cells and macrophages, the percentage of macrophages, as expressed in relation to the total number of cells in the composition, is from about 5% to about 70%, more preferably from about 20% to about 50%, and more preferably of about 35%.

In a more particular embodiment, a pharmaceutical composition of the invention contains macrophages wherein the percentage of macrophages, expressed in relation to the total number of cells in the composition, is from about 5% to about 65%.

In a particular embodiment, a pharmaceutical composition of the invention contains a first type of cells, possibly mixed with macrophages after the co-culture, frozen in aliquots and kept in suitable vehicle plus a cryopreservant at −80 to −130° C. and macrophages kept frozen in aliquots after culture. These tubes or bags containing the stem or precursors cells and the frozen macrophages can be thawed before injection into the lesion or in damaged tissues.

In another particular embodiment, a pharmaceutical composition of the invention contains frozen precursors cells or stem cells on one hand and frozen macrophages on other hand, in pharmaceutically acceptable cryopreservant and vehicle.

In a particular embodiment, a pharmaceutical composition of the invention contains myogenic precursor cells and macrophages.

In a more particular embodiment, a pharmaceutical composition of the invention contains myogenic precursor cells and macrophages wherein the ratio between macrophages and myogenic precursor cells, as expressed in number of cells, is comprised between about 1/10 and about 10/1, and preferably of about 1/1.

In a more particular embodiment, a composition according to the invention contains at least about 65% of myogenic precursor cells and macrophages, said percentage of myogenic cells plus macrophages being expressed in relation to the total number of cells present in the composition.

In a still more particular embodiment, a composition according to the invention between about 70 and 90% of myogenic precursor cells and macrophages. In another particular embodiment, a composition of the invention contains from about 35 to about 45% of macrophages and from about 35 to about 45% of myogenic precursor cells, said percentages being expressed in relation to the total number of cells present in the composition.

In another particular embodiment, a pharmaceutical composition of the invention contains myogenic precursor cells and macrophages wherein the percentage of cells, expressed in relation to the total number of cells in the composition, is comprised from about 10% to about 80% of macrophages, more preferably about 50%, and from about 10% to 80% of myogenic precursor cells, more preferably about 50%.

In a particular embodiment, a pharmaceutical composition of the invention contains myogenic precursor cells and macrophages wherein macrophages range from about 0.5 10⁸ to about 7.5 10⁸ and preferably from about 1.5 10⁸ to about 2.5 10⁸.

In a particular embodiment, a pharmaceutical composition of the invention contains myogenic precursor cells and macrophages wherein myogenic precursor cells range from about 0.5 10⁸ to about 7.5 10⁸ and preferably from about 1.5 10⁸ to about 2.5 10⁸ myogenic precursor cells.

The present invention also provides a binary complex made of a myogenic precursor cell and a macrophage, interacting by direct cell to cell contacts. Said binary complex being possibly observed by techniques known by a skilled person, such as histological observation. Said binary complex differs from a complex in which macrophages would phagocytose mpc.

In a particular embodiment, a binary complex according to the invention is characterized in that cell to cell contacts are mediated, at least partly, via cell surface molecules VLA4 and VCAM1, on the surface of myogenic precursor cells and macrophages. In another particular embodiment, a binary complex according to the invention is characterized in that cell to cell contact is mediated, at least partly, via fractalkine (CX3CL1) and CX3CR1 molecules, on the surface of myogenic precursor cells and macrophages.

Said cell to cell contacts are mediated by non-covalent specific interactions between the cell-surface molecules.

The present invention also provides a process for preparing pharmaceutical compositions containing a first type of cells and macrophages, comprising the steps of i) Preparing a first composition containing a first type of cells, chosen among the group consisting of precursor cells and stem cells (ii) preparing a second composition containing macrophages, (iii) contacting said first composition with said second composition. In a particular embodiment, said process is characterized in that said first composition and said second composition are contacted for a time sufficient to allow at least one cycle of cellular division.

The first and second composition are prepared according to techniques well known in the art to allow the correct handling and conservation of the first and second type of cells. In particular, cells are conserved in a medium compatible with their survival and/or proliferation. Said medium being possibly any medium appropriate for the ex vivo and in vivo cells survival or culture. Culture media of the type of HAM-F12 are preferably used, but any culture media convenient for efficient cell survival, culture, and possibly administration, is usable. Such process allows the ex vivo division of cells and cells to cells interactions, which may favor later engraftment of the precursor or stem cells contained in the composition.

The present invention also provides a product containing macrophages and a first type of cells, being possibly precursor cells or stem cells, as a combined preparation for the separate, simultaneous or sequential use in cellular graft into a mammal. In a particular embodiment, a product according to the invention contains macrophages and myogenic precursor cells.

In a particular embodiment, the product according to the invention where aliquots of the first type of cells and the macrophages are kept frozen in acceptable vehicle until thawing for the injection.

The present invention may for example find its application in substitutive cell therapy. In a particular embodiment, said substitutive cell therapy aims at replacing missing cardiomyocytes by contractile cells to repair damaged heart tissue. Focal muscle diseases constitutes choice candidates for said substitutive cell therapy.

### FIGURE LEGENDS

FIGS. 1A, 1B, 1C, 1D and 1E: In vitro human mpc myogenesis. FIG. 1A: mpc growth is expressed in number of cells/cm² (closed circles, left Y axis) and mpc differentiation is estimated by the fusion index (open circles, right Y axis). Mpc growth and differentiation related to days of culture. FIG. 1B: myogenin immunoblot at day 7, 14 and 21 of mpc culture. FIG. 1C, FIG. 1D, FIG. 1E: May-Grünwald Giemsa stain of mpc at day 7 (FIG. 1C), 14 (FIG. 1D) and 21 (FIG. 1E) of culture. x20 objective. FIGS. 1A, 1B, 1C, 1D and 1E put in evidence the augmentation of myogenesis during culture.

FIGS. 2A, 2B, 2C, 2D, 2E, 2F: Monocyte chemotaxis by mpc is specific and regulated during myogenesis. FIG. 2A: percentage of CD 14+ cells among PBMC (Y axis) before (upper chamber) and after (lower chamber) chemotaxis toward mpc-conditioned medium. Each circle represents one experiment and bars are means. FIG. 2B: monocyte chemotaxis (percentage of chemotaxis on Y axis) toward mpc-conditioned medium during myogenesis, related to days of culture (X axis). FIG. 2C: monocyte chemotaxis (percentage of monocyte chemotaxis on Y axis) normalized to 1×10⁵ cells, related to days of culture (x axis) (closed circle symbol). “Jurkat” (open square symbol) and “MCF-7” (open diamond symbol) relate to chemotactic activity exerted by Jurkat and MCF-7 cells respectively. FIG. 2D: fusion index (upper histogram) and normalized monocyte chemotaxis (lower histogram) of mpc cultured in standard (black bars) or differentiating conditions (white bars). Left group of bars correspond to “proliferating” stage (day 7 of culture), middle group of bars to “early fusion” stage (day 14) and right group of bars to “late fusion” stage (day 21). FIG. 2E: Monocyte chemotaxis (% of monocyte chemotaxis on y axis) along gradients of mpc-conditioned medium (day 14) at various concentrations in upper and lower chambers (x axis, from 0/2 to 0/0). FIG. 2F: monocyte chemotaxis (% of monocyte chemotaxis on y axis) toward mpc-conditioned medium (day 14) across HMVEC monolayer, related to mpc supernatant concentration, from 0.5× to 2×, on x axis. All results are means±SEM of at least 3 experiments run in triplicate. FIGS. 2A to 2F put in evidence different parameters of monocyte chemotaxis by mpc.

FIGS. 3A and 3B. Human muscle satellite cells are close by capillaries. Arrows show CD56+ satellite cell labeling, arrowheads show capillaries. CD56 is expressed at both membrane and cytoplasmic levels, as seen on satellite cells with rounded shape (FIG. 3B, upper right corner). x10 (FIG. 3A) and x40 (FIG. 3B) objective.

FIGS. 4A, 4B, 4C, 4D, 4E, 4F: Mpc constitutively express 5 monocyte chemotactic factors. FIG. 4A: RT-PCR analysis of mpc mRNA at day 14 of FKN (1), MDC (2), MCP-1 (3), VEGF (5). β2 microglobulin (4,6). FIG. 4B, FIG. 4C, FIG. 4D: Monocyte chemotactic factors in mpc supernatant (in pg/ml/1×10⁵ cells, on Y axis) as assessed by ELISA: measurement of MDC (FIG. 4B), MCP-1 (FIG. 4C) and VEGF (FIG. 4D). Each symbol (closed square, triangle and circle) represents one culture estimated in triplicate. X axis represents the days of culture. FIG. 4E, FIG. 4F, FIG. 4H: Immunolabeling of FKN (FIG. 4E), MDC (FIG. 4F), MCP-1 (FIG. 4G), VEGF (FIG. 4H) using FITC-conjugated secondary antibody. Blue: DAPI stain. x40 objective. FIGS. 4A to 4H represent the measured expression of each of the chemotactic factors.

FIG. 5. Five chemotactic systems ensure monocyte chemotaxis by mpc. Monocyte chemotaxis toward mpc-conditioned medium (% of monocyte chemotaxis on Y axis, day 14) was performed in the absence (none) or presence of whole mice and rabbit IgGs or antibodies directed against MCP-1, MDC, VEGF, FKN, CX₃CR1, uPAR, uPA. Results are means±SEM of 3 experiments run in triplicate. “All” corresponds to reaction the presence of all the previously cited antibodies.

FIGS. 6A to 6T: Activated satellite cells express the monocyte chemoattractants in vivo. Muscle biopsy from a patient with pure necrotizing myopathy was labeled for both CD56 (green, left column), a marker of satellite cells and regenerating muscle fibers, and chemoattractants (red, second column from left). Colocalisation appears in yellow in merging pictures (on black and white picture, merging signal appears as an increase white signal, third column from left). FIG. 6A to FIG. 6C: one activated satellite cell and two neighboring non-myogenic cells express MCP-1 whereas two regenerating fibers do not. FIG. 6D to FIG. 6F: three activated satellite cells and one neighboring non-myogenic cell express MDC whereas one regenerating fiber does not. FIG. 6G to FIG. 6I: one small regenerating fiber and one neighboring non-myogenic cell express FKN whereas another regenerating fiber does not. FIG. 6J to FIG. 6L: several cells, including activated satellite cells and possibly one non-myogenic cell with a large nucleus, presumably a macrophage, express VEGF at the level of a necrotic fiber. FIG. 6M to FIG. 6O: a myogenic cell strongly expresses uPAR. Blue: DAPI stain. x63 objective.

FIGS. 7A and 7B: Mpc and MP interplay to enhance monocyte chemotaxis. Monocyte chemotaxis by mpc (% of monocyte chemotaxis, on Y axis, day 14) (FIG. 7A) and by macrophage (FIG. 7B) stimulated or not by the other cell type. On each figure, left part represents the chemotaxis activity of mpc or macrophages alone, whereas right side represents respectively the chemotaxi activity of mpc stimulated by macrophages (FIG. 7A) or chemotaxi activity of macrophages stimulated by mpc (FIG. 7B). Each symbol (open square, circle, triangle, diamond) represents one experiment run in triplicate and variations are SD. Symbols on the left represent mpc. Symbols on the right represent mpc stimulated by MP. Thick bar represents mean. FIGS. 7A and 7B show that mpc and macrophages exert a reciprocal positive effect on chemotaxis activity on monocytes.

FIGS. 8A, 8B, 8C and 8D: Mpc:Macrophages cocultures stimulate mpc growth. FIG. 8A and FIG. 8B: co-culture of PKH26-labeled mpc with MP (1:1 ratio) for 2 days shows absence of fluorescence in MP cytoplasms (circles). x20 objective. FIGS. 8A and 8B show the absence of mpc phagocytosis by macrophages. FIG. 8C and FIG. 8D: Density of mpc in direct (FIG. 8C) or indirect (FIG. 8D) co-cultures with MP at various seeding ratio. X axis represents days of culture, whereas Y axis represents mpc density (in cell/cm2). Mpc/MP ratios are 1/0 (open circle), 1:0.5 (closed square), 1:1 (closed triangle), 1:2 (closed diamond), 1:5 (black cross), 1:10 (closed circle). Results are means±SEM of 3 experiments run in duplicate.

FIGS. 9A and 9B: MP-secreted factors enhance mpc proliferation. [³H]-thymidine incorporation (y axis) of mpc treated with MP-conditioned medium (FIG. 9A) or co-cultured with MP (FIG. 9B). X axis in FIG. 9A represents the number of macrophages in supernatants, whereas X axis in FIG. 9B represents mpc/MP ratio with mpc alone (equivalent to mpc/MP ratio of 1:0), MP alone (equivalent of mpc/MP ratio of 0/1), and mpc/MP ratio of 1;0.5, 1:1 and 1:2. Each open symbol (circle, square, triangle, diamond and cross) represents one separate experiment run in triplicate and closed circles represent means±SEM.

FIGS. 10A, 10B, 10C and 10D: MP rescue mpc from apoptosis. FIG. 10A: oligosomal DNA measurement in mpc cultures, mpc treated with MP-conditioned medium, MP and mpc:MP cocultures. Results are means±SD of 3 experiments run in duplicate. In FIG. 10A, x axis represents the level of oligosomal DNA, as expressed by Optical Density; from left to right, histograms represent conditions with either mpc alone (white histogram), mpc with MP conditioned medium (gray histogram), macrophages alone (black histogram), theorical level including mpc alone and MP alone (white histogram superposed to black histogram) or mpc/MP cocultures (striated histogram). FIG. 10B to FIG. 10D: Cells were co-labelled with annexin and anti-CD56 antibodies, Mpc were cultured alone (FIG. 10B) or with MP (FIG. 10C) and labeled with annexin V (green) and anti-CD56 antibodies (red). FIG. 10B, FIG. 10C: Blue: DAPI stain. x60 objective. FIG. 10D: quantification of apoptotic cells among MP (black symbols) and mpc (white symbols) populations. Each symbol (circle, square and triangle) represents one separate culture. The percentage of apoptotic cells (y axis) is lower when cells are co-cultivated (right part of the graph) than in separate cultures (left part).

### ABBREVIATION LIST

FKN: fractalkine
HGF: hepatocyte growth factor
HMVEC: human adult microvascular endothelial cells
MCP-1: monocyte chemoattractant protein-1
MDC: macrophage-derived chemokine
MP: macrophage
mpc: myogenic precursor cells
PBMC: peripheral blood mononuclear cells
uPA: urokinase
uPAR: urokinase type plasminogen-activator receptor

### EXAMPLES

### Example 1

### Attraction of Monocytes by Human Myogenic Precursor Cells (Mpc)

A. Material and Methods

Cell cultures. Unless indicated, culture media components were from Gibco (Paisley, Scotland) and culture plastics from TPP AG (Trasadingen, Switzerland).

Human mpc were cultured from muscle samples as previously described {Bonavaud, Thibert, et al. 1997 542/id}. In standard culture conditions (spontaneous in vitro myogenesis) mpc were grown in HAM-F12 medium containing 15% FCS (growing medium) without serum withdrawal. In differentiating conditions, growing medium was replaced by HAM-F12 medium containing 5% FCS (differentiating medium) at time of subconfluence. Only cultures presenting over 95% CD56+ (1/20, 123C3, Sanbio/Monosan, Uden, Netherlands) cells were used.

PBMC isolated from human blood using Ficoll Paque plus (Pharmacia Biotech, Piscataway, N.J.) density gradient were immediately used. Monocytes isolated from PBMC by an adhesion step {Gruss, Brach, et al. 1994 211/id} were immediately used. Purity, estimated by flow cytometry after CD45-FITC (KC56(T-200) Coulter, Miami, Fla.) and CD14-PE (RM052, Immunotech, Marseille, France) labeling, ranged from 80 to 90%.

To obtain MP, monocytes were seeded at 0.5×10⁶ cell/ml in Teflon bags (AFC, Gaithersburg, Md.) in differentiating RPMI medium containing 15% human AB Serum for 8 days {Gruss, Brach, et al. 1994 211/id} {van der Meer, van de Gevel, et al. 1982 218/id}.

Jurkat cells were grown in RPMI containing 10% FCS. MCF-7 cells were grown in DMEM containing 5% FCS and 1% non-essential amino acids.

Mpc:MP coculture ratio ranged from 1:0.5 to 1:10. For growth and immunolabeling experiments, 1=2000 cells/cm². In indirect cocultures, MP were seeded in inserts (0.4 μm diameter pores) (Falcon, BD Biosciences, Franklin Lakes, N.J.) placed over the mpc-containing well. In 96-well plates (proliferation and apoptosis assays), 1=30000 cells/well.

Conditioned media. Conditioned media were obtained by incubating cells in 24-well plates in serum-free HAM-F12 for 24 h in 0.5 ml (1×). For chemotaxis assay at constant cell/supernatant ratio, the volume of supernatant was adjusted exactly to the mpc number (300 μl for 10000 mpc). 2× and 3× concentrations of mpc-conditioned media were obtained by proportional reduction of medium in mpc culture. Cell stimulation by conditioned medium was performed by incubating cells for 30 h with medium conditioned the day before.

Mpc growth and differentiation. Mpc density was determined by counting cells after trypsinization. Trypsin treatment did not detach MP, as flow cytometry analysis of detached cells after CD64-FITC (10.1, Pharmingen, BD Biosciences) and CD14-PE labeling showed no CD14+ and less than 0.4% CD64+cells. Fusion index was calculated as described before {Authier, Chazaud, et al. 1999 351/id}. Myogenin immunoblotting was carried out using 40 μg mpc protein extract and M-225 antibody (1:200) (Santa Cruz Biotechnology, Santa-Cruz, Calif.) {Fujio, Guo, et al. 1999 549/id}.

Chemotaxis. Leukocytes (500000 in serum-free HAM-F12 medium) were deposited into Falcon insert (3 μm diameter pores) put on top of a well containing conditioned medium and plates were incubated at 37° C. for 24 h. The number of cells present in the well was evaluated and expressed as percentage of number of deposited cells. Chemotaxis toward HAM-F12 medium was considered as non-specific chemotaxis, which value was subtracted from observed values. No leukocyte was present at the insert lower face. In some experiments, blocking antibodies were added in the well at saturating concentrations (calculated from IC50 or from previous studies): anti-MCP-1 (3 μg/ml, P500-P34, Abcys, Paris, France), anti-FKN (3 μg/ml, 51637.11, R&D Systems, Minneapolis, Minn.), anti-MDC (6 μg/ml, AF336, R&D), anti-VEGF (6 μg/ml, AF293NA, R&D), anti-CX₃CR1 (15 μg/ml, Torrey Pines Biolabs, Houston Tex.) {Feng, Chen, et al. 1999 706/id}, anti-uPA (4 μg/ml, #394, American Diagnostica, Greenwich, Conn.), anti-uPAR (5 μg/ml, #3936, American Diagnostica) {Chazaud, Ricoux, et al. 2002 95/id}. Controls included addition of whole mice and rabbit IgGs (3 μg/ml each, Vector Laboratories, Burlingame, Calif.).

Transendothelial chemotaxis was performed using human adult microvascular endothelial cells (HMVEC) cultured according to the manufacturer's instructions (Biowhittaker, Walkersville, Md.). HMVEC were seeded at 10000 cells/cm² in Falcon inserts (3 μm diameter pores). Three days after confluence, HMVEC monolayer integrity was assessed in 2 wells by absence of Trypan blue (0.2% in 0.1% BSA) translocation from upper to lower chamber after 3 h incubation. HMVEC were incubated overnight with conditioned medium in the lower chamber before monocytes were added in the upper chamber. The number of monocytes present in the medium of the lower chamber was determined after 24 h. No monocyte was present at the insert lower face.

Statistical analysis. Excepted DNA array, all experiments were performed using at least 3 different cultures. The student t test was used for statistical analyses. A P value<0.05 was considered significant.

B. Results

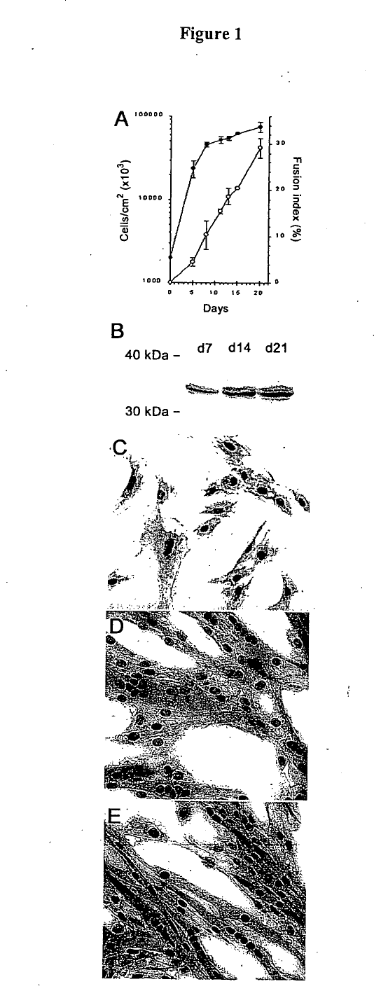

Human mpc in culture were at the stage of proliferation (day 7), early differentiation (day 14), and late differentiation (day 21) when grown without serum withdrawal (FIG. 1). In these conditions, all mpc do not achieve full differentiation. However, myogenesis was assessed by both myotube formation, the fusion index reaching 30% at day 21, and increasing myogenin expression (FIG. 1).

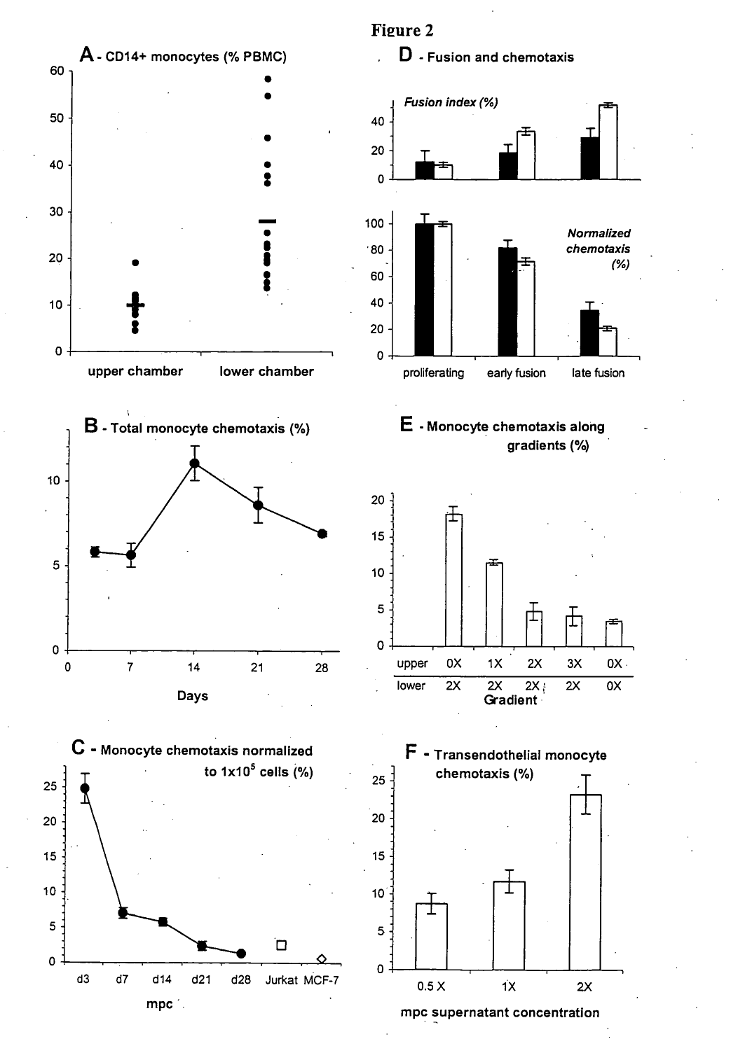

In conventional chemotaxis assays, human mpc attracted PBMC, 5% of PBMC being specifically attracted at day 7, 9% at day 14, and 6% at day 21 (day 7 vs. 14 increase: p<0.003, day 14 vs. 21 decrease: p<0.05). Chemotaxis selectively involved monocytes as assessed by enrichment of the attracted cells in CD14+ cells (28% vs. 10%, p<0.0001) (FIG. 2A). Enrichment in CD14+ cells was similar at all stages of mpc culture. It was not due to modulation of CD14 expression by the mpc-conditioned medium.

Similarly to PBMC, isolated human monocytes were attracted by mpc with a peak of chemotaxis at day 14 (day 7 vs. 14 increase: p<0.0001, day 14 vs. 21 decrease: p<0.005) (FIG. 2B). Because chemotactic activity of a differentiating cell population may reflect both the state of differentiation and the number of cells at each time point, we calculated chemotaxis normalized for 1×10⁵ mpc at each time point. FIG. 2C shows that normalized mpc chemotactic activity was high at day 3, dropped at day 7, and progressively declined at subsequent stages of differentiation. Differentiated myotubes exhibited a low normalized chemotactic activity similar to that of other cell types, including Jurkat and MCF-7 cells. In standard culture conditions, the volume of medium remains constant at each time point. To avoid bias in calculation of normalized mpc chemotactic activity due to variations of medium volume/cell number ratio, we measured chemotaxis at a constant ratio. Day 3 and 7 time points exhibited the highest difference of volume/cell number ratio in standard conditions. Chemotaxis measured at a constant ratio showed a decrease by 42% of mpc chemotaxis from day 3 to 7 (P<0.005), confirming that mpc exhibit maximal individual chemotactic activity shortly after their release from quiescence. A similar experiment conducted at day 7 and 14 revealed a decline of chemotaxis by 18%, very close to that obtained by calculation (19%).

Normalized chemotaxis of mpc grown in differentiating conditions to stimulate myotube formation showed a more abrupt decrease than that of mpc allowed to spontaneously differentiate (FIG. 2D), confirming that mpc differentiation is associated with a decline of their chemotactic activity.

The monocyte attraction was shown to be directional by varying mpc-conditioned medium concentrations in the chemotaxis chambers. Increasing gradients from the upper to the lower chamber induced migration of monocytes, but neither absent nor reverse gradients did (FIG. 2E). Moreover, chemotaxis correlated positively with the gradient magnitude (FIG. 2E).

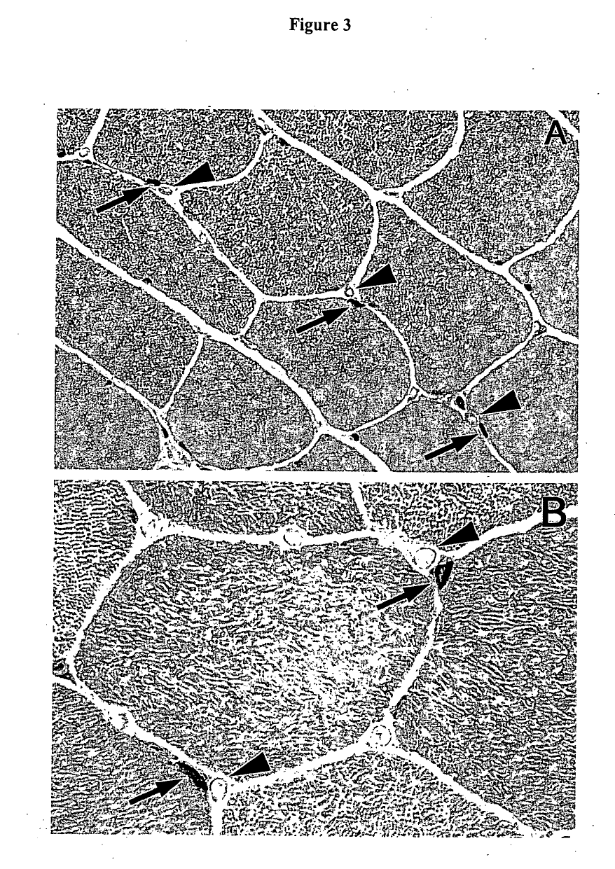

Microvessel-derived endothelial cells were used to control that mpc chemotaxis remains operative across an endothelial layer. Using various mpc supernatant concentrations, a dose-dependent transendothelial monocyte migration (p<0.05) was observed (FIG. 2F). This assay approximated the in vivo situation as demonstrated by microanatomic study of human adult muscle. As shown in FIG. 3, a majority of CD56+ satellite cells were located close by capillaries (87% being 5-20 μm from a capillary). The mean distance from a satellite cell nucleus to the closest capillary lumen center was 12.7±7.5 μm.

These results show that human mpc can selectively and specifically attract monocytes through an endothelial layer in a dose-dependent fashion. This previously unreported property of mpc varied according to the differentiation stage, individual chemotactic activity of satellite cells being high shortly after their release from quiescence and then declining progressively to reach levels similar to that of other cell types at time of late differentiation into multinucleated myotubes.

### Example 2

### Identification of a Set of Candidate Chemotactic Factors in Mpc and Determination of the Main Effectors

A. Material and Methods

DNA Array. Total RNA was prepared from mpc at day 7 and 14 of culture using the RNeasy mini kit (Qiagen, Hilden, Germany). All further steps (polyA enrichment, reverse transcription, ³²P-labeling and membrane hybridization) were performed according to the manufacturer's instructions in the Atlas Human Hematology/Immunology Array (#7737-1) (Clontech, BD Biosciences) kit. For day 7 and 14 samples, 9 and 7 μg of total RNA gave roughly similarly labeled cDNA: 989000 and 963000 cpm, respectively, that were deposited on membranes. Results were read using a Phosphorimager (Amersham, Buckinghamshire, UK) after a 4 day exposure time. Analysis was performed using Image Quant software (Amersham), that allows background noise subtraction, correction for the variation of density for housekeeping genes (all genes showed the same intensity variation between the 2 membranes), and finally, comparison of densitometric signals. Results were expressed in arbitrary units.

RT-PCR. Total mpc RNA (2 μg) was reverse transcribed and amplified using OneStep RTPCR (Qiagen) and specific primers. For FKN (primers in {Lucas, Chadwick, et al. 2001 2/id}) and MDC (primers in {Katou, Ohtani, et al. 2001 707/id}), amplification was performed at 94, 64 and 72° C. for 30 s, 30 s and 1 min, respectively, for 38 cycles. For MCP-1 (GenBank # M24545), the sense primer used was 5′-CCC AGT CAC CTG CTG TTA T-3′ and the antisense primer was 5′-AAT TTC CCC AAG TCT CTG TAT CTA-3′, amplification was performed at 94, 55, and 72° C. for 30 s for 38 cycles. For VEGF (primers in {Bausero, Ben Mahdi, et al. 2000 708/id}), amplification was performed at 94, 60 and 72° C. for 30 s, 30 s and 45 s, respectively, for 45 cycles. Amplification products (10 μl) were subjected to electrophoresis on 2% agarose and stained with ethidium bromide for visualization.

ELISA. MCP-1 (Coulter), MDC (R&D) and VEGF (Cytimmune Sciences Inc, College Park, Md.) concentrations in mpc-conditioned medium were determined by ELISA. ELISA for FKN was conducted as previously described {Foussat, Bouchet-Delbos, et al. 2001 705/id}. Results were corrected according to the cell number and are expressed in pg/ml for 1×10⁵ cells.

Mpc labelings. Mpc were labeled with primary antibodies for 2 h: anti-MCP1 (10 μg/ml), anti-FKN (50 μg/ml), anti-MDC (10 μg/ml), anti-VEGF (10 μg/ml), revealed using FITC-conjugated secondary antibody (1/100, Jackson Immunoresearch Laboratories, West Grove, Pa.) or biotin-conjugated secondary antibody (1/150, Jackson) and FITC-streptavidin (1/50, Vector).

Cells were labeled with annexin-V-biotin (Pharmingen) revealed by streptavidin-FITC (Jackson), and further labeled with anti-CD56 antibody (1/20) revealed using a goat anti-mouse TRITC antibody (1/100, Jackson). At least 100 cells from randomly chosen fields (x40 objective) were evaluated for their labeling.

Coverslips were mounted in vectashield containing DAPI (Vector). Controls included incubation with whole IgGs from species of the secondary antibody (50 μg/ml, Vector).

B. Results

A mRNA profiling technique, allowing analysis of a huge number of genes at once, was used. Among the 588 genes represented on the DNA macroarray membrane used, 20 had products known to attract monocytes, of which 5 were constitutively expressed by human mpc at day 7 and 14 of culture (Table 1): monocyte chemoattractant protein-1 (MCP-1, CCL2) {Zachariae, Larsen, et al. 1998 500/id}, macrophage-derived chemokine (MDC, CCL22) {Mantovani, Gray, et al. 2000 765/id}, fractalkine (FKN, CX₃CL1) {Bazan, Bacon, et al. 1997 763/id}, VEGF {Sawano, Iwai, et al. 2001 668/id}, and urokinase receptor (uPAR) {Resnati, Guttinger, et al. 1996 135/id}.

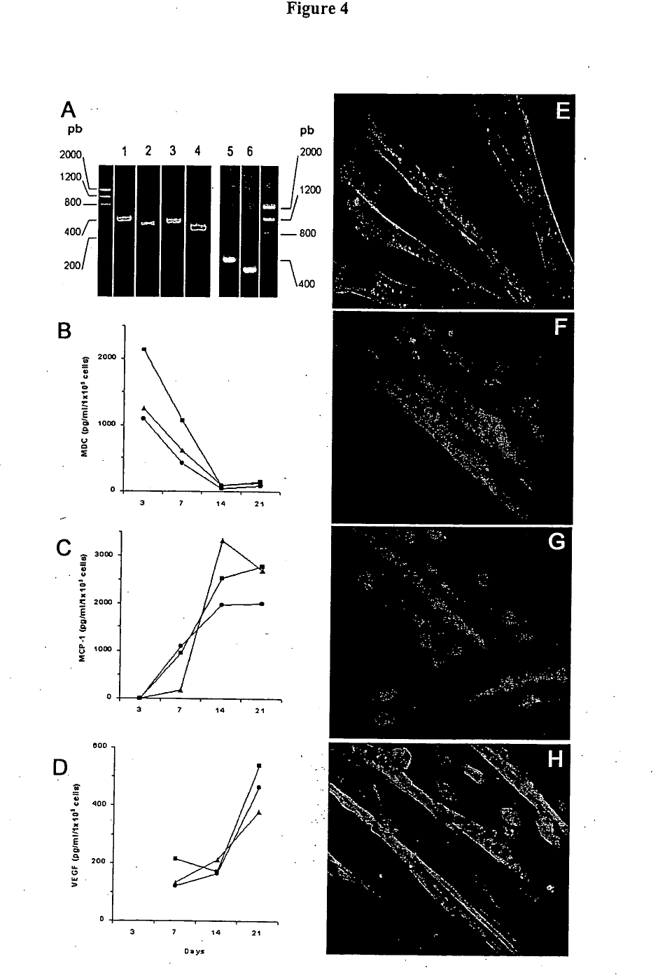

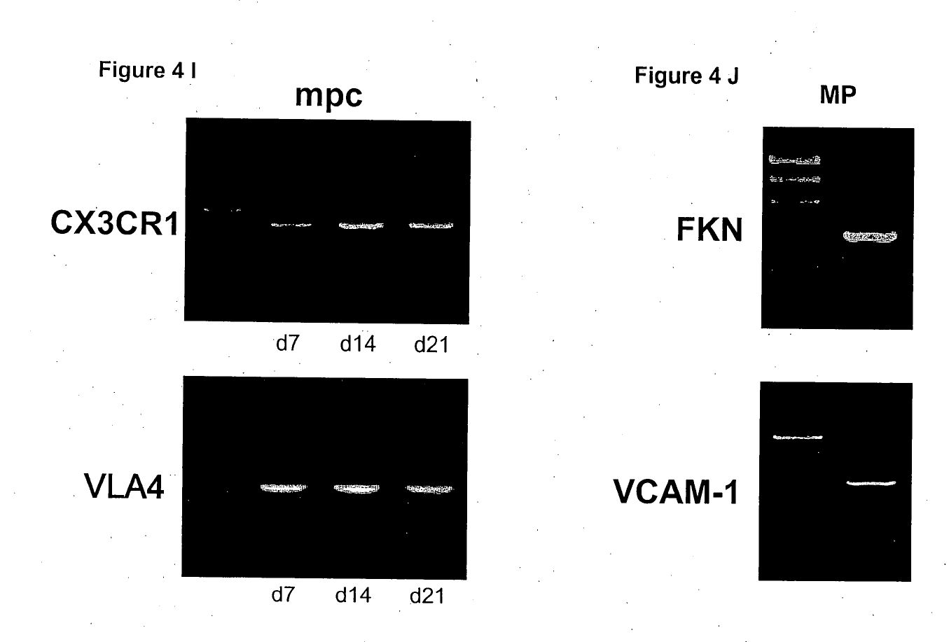

Confirmatory RT-PCR showed amplification products of MCP-1, MDC, FKN and VEGF transcripts at the expected molecular weight (FIG. 4A) in mpc culture. Both expression and upregulation of a functional uPAR/urokinase (uPA) chemokine-like system during human mpc differentiation were previously reported {Quax, Frisdal, et al. 1992 254/id} {Bonavaud, Charrière-Bertrand, et al. 1997 200/id} {Chazaud, Bonavaud, et al. 2000 449/id}. Constitutive mpc release of MCP-1, MDC and VEGF was assessed by ELISA. MDC level was high at day 7 and dropped at later stages (p<0.05) (FIG. 4B). MCP-1 levels markedly increased in mpc supernatant at time of fusion (p<0.02) (FIG. 4C), consistently with the 3-fold increase of MCP-1 mRNA level detected by DNA macroarray at day 14. VEGF level variations exhibited a strong increase at day 21 (p<0.005) (FIG. 4D). Soluble FKN levels did not reach the high detection threshold (70 pg/ml) of the ELISA we used {Foussat, Bouchet-Delbos, et al. 2001 705/id}. However, immunofluorescence confirmed cellular expression of FKN and the 3 other chemokines (FIG. 4E-H). Labeling of multinucleated cells unequivocally assessed a myogenic cell expression. Cytoplasmic immunopositivity was observed for all molecules. In addition, marked cell membrane labeling was observed for FKN (FIG. 4E).

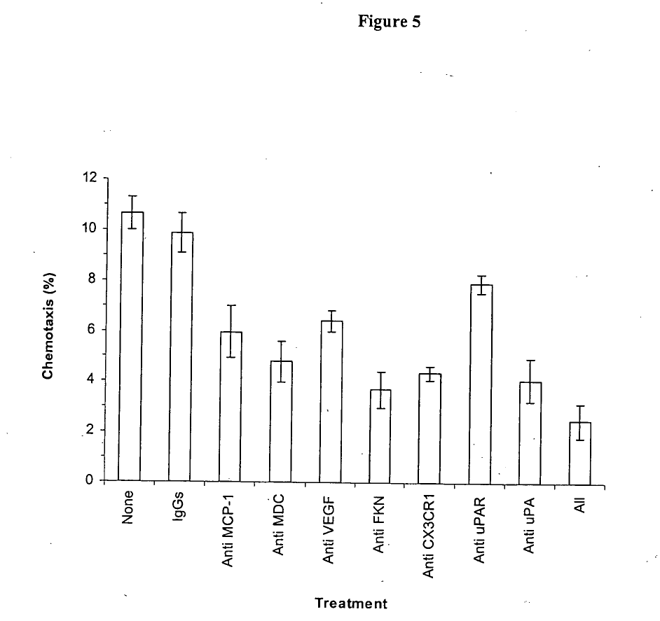

Functional involvement of the detected molecules was assessed using specific blocking antibodies (FIG. 5). Monocyte chemotaxis decreased by 45% after MCP-1 inhibition (p<0.01), 50% after MDC inhibition (p<0.005), 62% after FKN inhibition (p=0.003), 44% after VEGF inhibition (p<0.02) and 26% after uPAR inhibition (p<0.02). Whole Igs induced no effect. The presence of soluble FKN was assessed by blocking the cognate receptor CX₃CR1 on monocytes, which inhibited chemotaxis by 59% (p<0.005). To further inhibit the complicated mechanism underlying the uPAR/uPA chemokine-like effect, uPA, a strategy previously proved efficient {Resnati, Guttinger, et al. 1996 135/id}, was targeted. uPA inhibition induced a 58% decrease of chemotaxis (p<0.003). Since leukocytes integrate the various chemoattractant signals they receive through multiple and promiscuous receptors in a complex and still poorly understood fashion {Foxman, Campbell, et al. 1997 507/id}, the effect of global effector inhibition was analyzed. Pooling blocking antibodies against MCP-1, MDC, FKN, VEGF, uPAR and uPA induced a 77% inhibition of monocyte chemotaxis (p<0.03) (FIG. 5).

Mpc were shown to produce 5 monocyte chemoattractants accounting for 77% of chemotaxis at day 14 of culture. They included 3 chemokines, MDC, MCP-1 and FKN, one growth factor, VEGF, and one proteolytic system with chemotactic activity, uPA/uPAR.

Different profiles of secretion were observed for MDC that was mainly detected at day 7, MCP-1 that increased from day 14, and VEGF that increased at day 21.

The recently identified CC-chemokine MDC is not detected in normal human adult skeletal muscle {Mantovani, Gray, et al. 2000 765/id}. It functions through the CCR4 receptor, which is expressed by 6% of human monocytes {Katschke, Rottman, et al. 2001 771/id}, and at least another important, as yet unknown, receptor {Mantovani, Gray, et al. 2000 765/id}. In addition to its chemotactic effect on monocytes, MDC activates MP and enhances their phagocytic activity more rapidly than does MCP-1, in vivo {Matsukawa, Hogaboam, et al. 2000 773/id}. Thus, MDC likely represents an early mpc-delivered signal for monocyte recruitment and MP activation.

The CC-chemokine MCP-1 is produced, mainly under proinflammatory conditions, by a large variety of cells {Zachariae, Larsen, et al. 1998 500/id}. CCR2 receptor, that is expressed by 71% of human monocytes {Fantuzzi, Borghi, et al. 1999 606/id}, mediates MCP-1 effects on monocyte chemotaxis and activation {Zachariae, Larsen, et al. 1998 500/id}. Constitutive myogenic cell expression of MCP-1 was previously reported in rat {Reyes-Reyna & Krolick 2000 741/id} and human rhabdomyosarcoma {Astolfi, De Giovanni, et al. 2001 775/id} cell lines, but not in primary human mpc cultures {De Rossi, Bernasconi, et al. 2000 630/id}. Interestingly, we did not detect MCP-1 transcripts by RT-PCR at day 3 (data not shown), a very early stage of culture, although a unique upregulation of MCP-1 mRNA expression was documented at subsequent stages. Such a differentiation-associated upregulation of MCP-1 expression was previously reported in both a rhabdomyosarcoma cell line {Astolfi, De Giovanni, et al. 2001 775/id} and monocytes/MP {Gruss, Brach, et al. 1994 211/id} {Fantuzzi, Borghi, et al. 1999 606/id}. Upregulation of MCP-1 production is pivotal for amplification of chemotaxis {Cushing & Fogelman 1992 776/id} {Andjelkovic, Kerkovich, et al. 2000 602/id}. Thus, MCP-1 appears as a secondary signal for monocyte recruitment and MP activation, delivered by mpc at time of MDC downregulation in the setting of chemotaxis amplification.

VEGF induces vascular cell chemotaxis, survival, and proliferation, mainly through VEGF-R2 {Rissanen, Vajanto, et al. 2002 401/id}. Among its non-vascular roles, VEGF is chemotactic for monocytes through VEGF-R1, a receptor expressed by 83% of human monocytes {Sawano, Iwai, et al. 2001 668/id}. Muscle fiber expression of VEGF and VEGF-R2 is induced by ischemia {Rissanen, Vajanto, et al. 2002 401/id}. It is associated with focal MP infiltration and vessel hyperplasia and might prevent muscle cell death and support regeneration {Rissanen, Vajanto, et al. 2002 401/id}. Similar VEGF effects may be at play after other types of muscle injury, as well.

The CX₃C chemokine FKN contains a chemokine domain fused to a mucin-stalk tethered to a transmembrane domain with an intracytoplasmic tail {Bazan, Bacon, et al. 1997 763/id}. FKN transcripts have been previously detected in normal human muscle homogenates {Bazan, Bacon, et al. 1997 763/id}. In FKN-producing cells, such as endothelial cells, 90% of FKN is membrane bound at steady state and 10% is cleaved in a soluble form {Imaizumi, Matsumiya, et al. 2000 777/id}. Soluble FKN is angiogenic {Volin, Woods, et al. 2001 180/id} and chemotactic for monocytes {Bazan, Bacon, et al. 1997 763/id} {Chapman, Moores, et al. 2000 164/id} through the cognate receptor CX₃CR1, that is expressed by 56% of human monocytes {Ruth, Volin, et al. 2001 769/id}. In our study, both anti-FKN and anti-CX₃CR1 antibodies inhibited mpc chemotactic activity but FKN could not be detected in supernatants by ELISA. This was in keeping with previous evidence that attraction of human monocytes by FKN may occur at concentrations far below the ELISA detection threshold {Chapman, Moores, et al. 2000 164/id}.

The uPA system mainly includes the receptor uPAR, its ligand uPA and the matrix-bound inhibitor PAI-1 {Preissner, Kanse, et al. 2000 111/id}. The three components are markedly upregulated during muscle regeneration {Lluis, Roma, et al. 2001 676/id} {Festoff, Reddy, et al. 1994 787/id} and at time of fusion in human mpc cultures {Chazaud, Bonavaud, et al. 2000 449/id} {Bonavaud, Charrière-Bertrand, et al. 1997 200/id} {Quax, Frisdal, et al. 1992 254/id}. uPA activates Hepatocyte Growth Factor (HGF) through cleavage of its matrix-associated inactive precursor {Naldini, Tamagnone, et al. 1992 501/id}, which might trigger activation of quiescent satellite cells through c-met, the HGF receptor {Allen, Sheehan, et al. 1995 57/id}. In addition, the uPA system exerts proteolytic and non-proteolytic roles operative in cell migration {Preissner, Kanse, et al. 2000 111/id} {Chazaud, Bonavaud, et al. 2000 449/id}. A soluble form of truncated uPAR, present in body fluids {Sidenius, Sier, et al. 2000 784/id}, mediates chemotaxis of myelomonocytic cells by inducing signal transduction through an unknown transmembrane adaptor {Resnati, Guttinger, et al. 1996 135/id}. uPA exerts similar chemotactic effects through uPAR and the same unknown adaptor {Resnati, Guttinger, et al. 1996 135/id}. In our system, uPAR blockade could not assess the proper role of soluble uPAR since it interfered with uPA:uPAR binding at the membrane of monocytes. Consistently, anti-uPA antibodies induced inhibition of chemotaxis. A crucial role of uPA in muscle regeneration was demonstrated in uPA deficient mice {Lluis, Roma, et al. 2001 676/id}, and reflects the multifunctional status of the uPA system that could control satellite cell activation, monocyte chemotaxis and mpc migration {Chazaud, Bonavaud, et al. 2000 449/id}.

### Example 3

### In Vivo Expression of Monocyte Chemoattractants by Activated Satellite Cells

A. Material and Methods: Human Muscle Immunohistochemistry.

Cross sections of frozen adult deltoid muscle biopsy samples were labeled with mouse anti-human CD56 (1/20, NHK-1-RD1, Coulter) revealed using peroxidase Vectastain ABC kit (Vector). The distance from the CD56+ satellite cell nucleus to the lumen center of the nearest capillary was determined on 50 satellite cells in randomly chosen fields using the KS300 Imaging software (Carl Zeiss Vision, Hallbergmoos, Germany).

For double labeling, sections were labeled with mouse anti-human CD56 revealed by goat anti-mouse-FITC (1/100, Jackson) and were further labeled for MCP1, FKN, MDC, VEGF and uPAR, revealed using TRITC-conjugated antibodies as described above.

B. Results

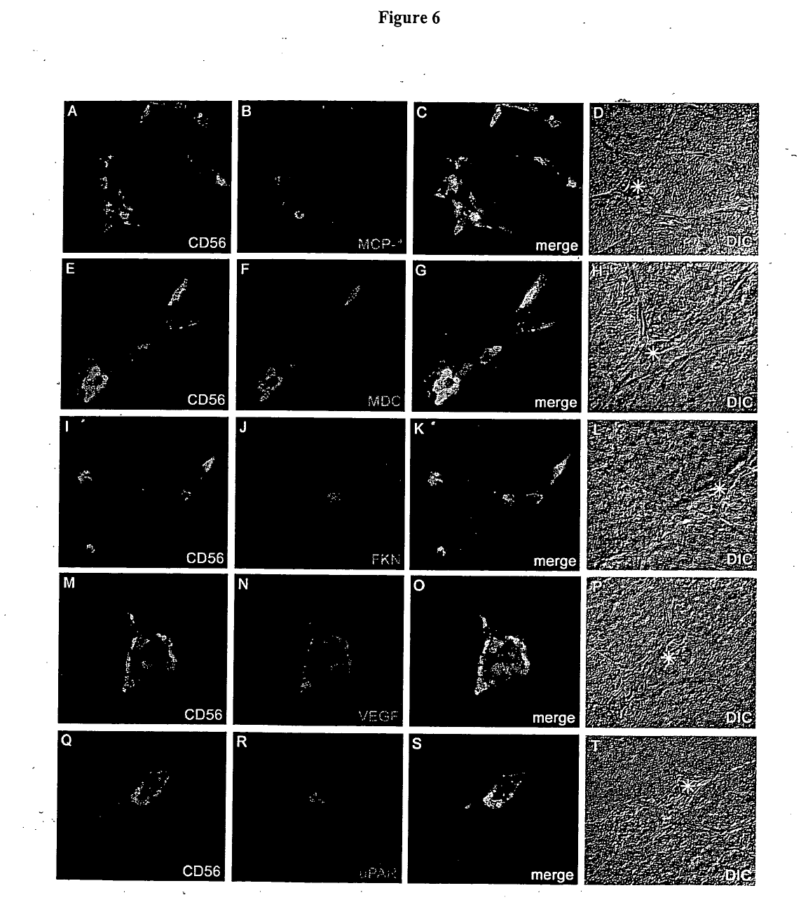

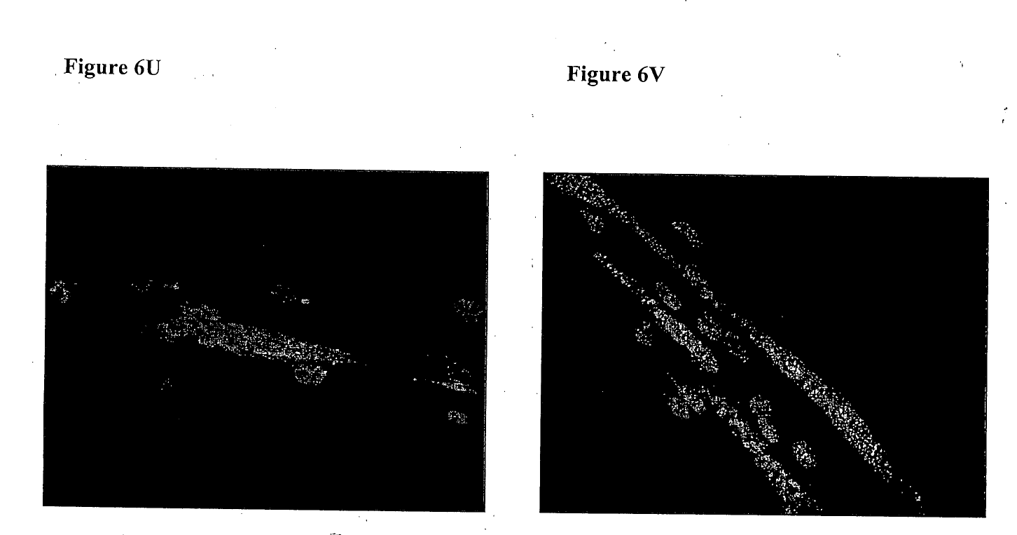

In vivo relevance of the previously cited findings was assessed by double immunostaining for CD56 and each effector on cryosections of a muscle biopsy showing pure necrotizing myopathy, i.e. patchy degeneration/regeneration without lymphocytic infiltrates. Diseased areas showed unambiguous satellite cell MCP-1, MDC, FKN, VEGF and uPAR expression (FIG. 6). Muscle fibers expressing chemoattractants were rare and always co-expressed CD56, a marker of regeneration. Immunopositivities were observed in non-myogenic mononuclear cells, within necrotic fibers (FIG. 6J-L) or in the interstitial tissue close to activated satellite cells (FIG. 6). Chemoattractants were not expressed in normal-looking muscle areas.

### Example 4

### Mpc/Macrophage Interaction

A. Material and Methods

To obtain macrophages (MP), monocytes were seeded at 0.5×10⁶ cell/ml in Teflon bags (AFC, Gaithersburg, Md.) in differentiating RPMI medium containing 15% human AB Serum for 8 days {Gruss, Brach, et al. 1994 211/id} {van der Meer, van de Gevel, et al. 1982 218/id}.

Cell proliferation. Mpc were cultured with MP in HAM-F12 medium, or with MP-conditioned medium containing [³H]-thymidine (1 μCi/ml) for 18 h. Trypsin-EDTA (50 μl) was added, radiolabeled DNA was recovered on MultiScreen Harvest plates (Millipore, Bedford, Mass.) using a manual Harvester (PerkinElmer, Boston, Mass.) and quantified in a beta counter.

Oligosomal DNA levels. Mpc were cultured with macrophages (MP) in HAM-F12 medium, or with MP-conditioned medium for 18 h, and treated using the Cell Death Kit (Roche Diagnostic, Mannheim, Germany).

B. Results

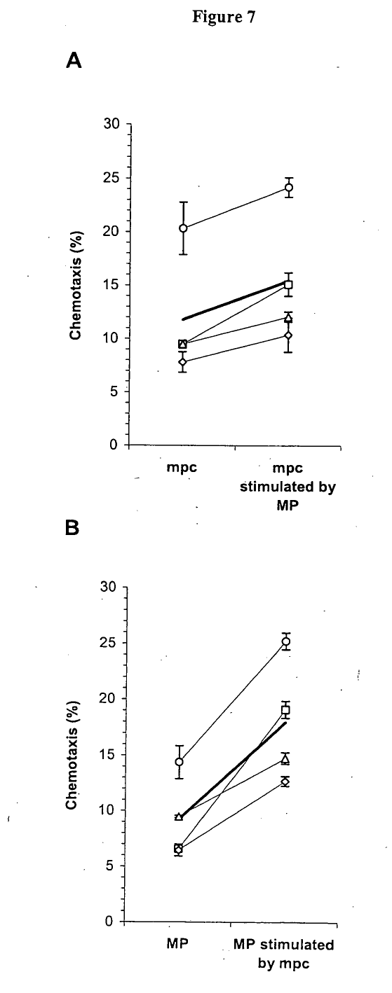

As compared to classically cultured mpc, mpc incubated 30 h with MP-conditioned medium increased by 31% their chemotactic effect on monocytes (p<0.02) (FIG. 7A). The factors involved in constitutive mpc chemotaxis were also implicated here since global inhibition decreased monocyte chemotaxis by 67% (p<0.006) (data not shown). MP stimulation of mpc chemotaxis was specific, since it was not reproduced by mpc-conditioned medium (p=0.62, data not shown). Conversely, MP incubated with mpc-conditioned medium increased their chemotaxis by 94% (p<0.02) (FIG. 7B). This stimulation was not reproduced by MP-conditioned medium (p=0.80, data not shown).

MP Stimulate Mpc Growth

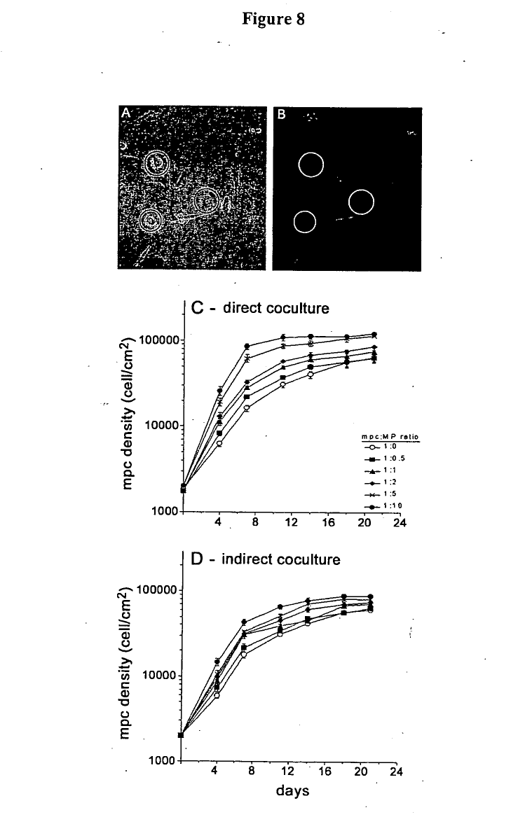

Cocultures at various mpc:MP ratios were performed to further evaluate cell interplays. We first examined if MP operate phagocytosis of PKH26-labeled mpc. No intracytoplasmic fluorescent signal was observed in MP after 1 to 4 days of coculture, whatever the cell ratio (ranging from 1:0.5 to 1:2), ruling out significant phagocytosis of living mpc by MP (FIG. 8A-B).

Mpc growth curves were established under culture conditions allowing, or not, direct mpc:MP contacts. MP induced a dose-dependent increase of mpc density in both conditions, but enhancement was stronger in conditions allowing mpc:MP contacts (FIG. 8C) than in cultures separated by a porous filter (FIG. 8D) (5.3 fold vs. 2.4 fold increase of mpc density at day 7 of culture at the 1:10 [mpc:MP] ratio, p<0.02).

MP Promote Mpc Proliferation by Soluble Factors and Mpc Survival by Direct Contacts

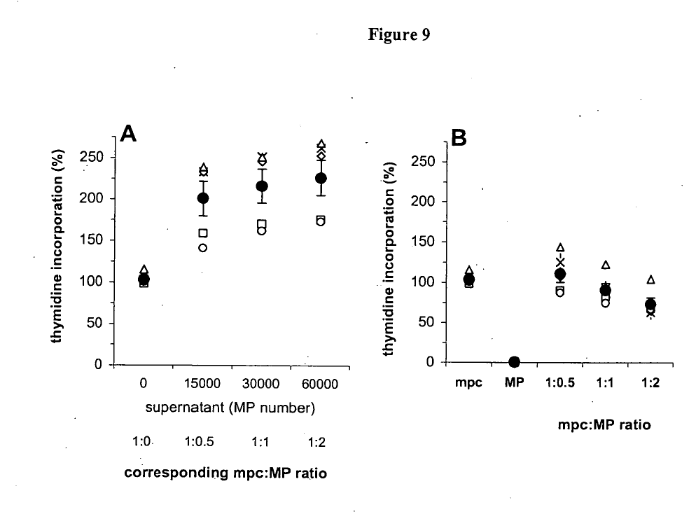

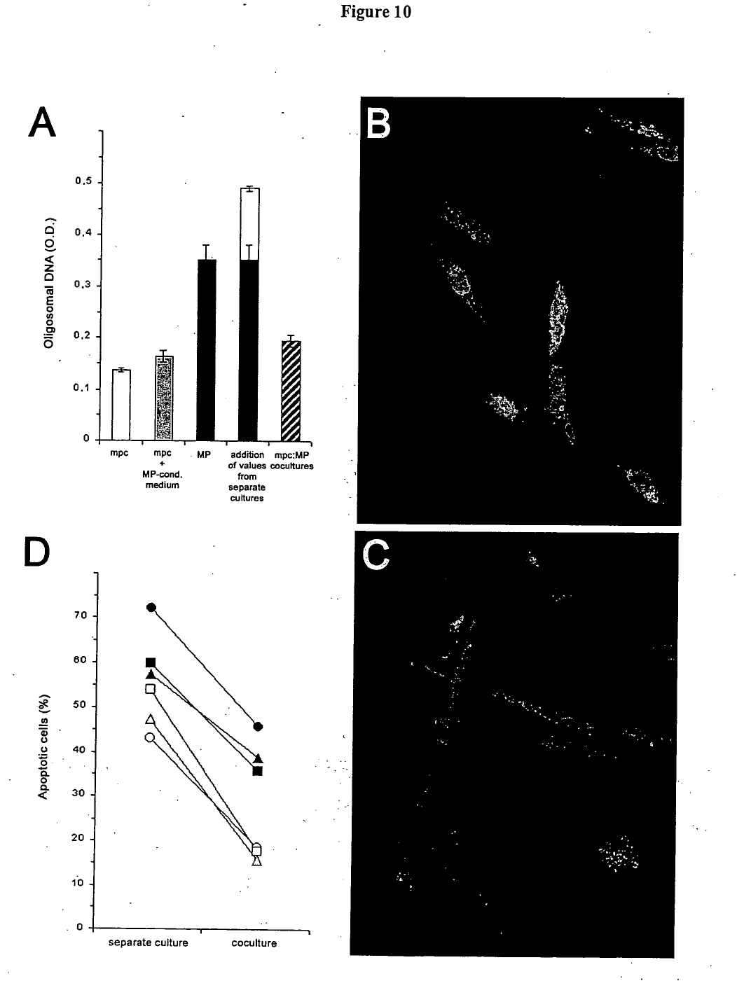

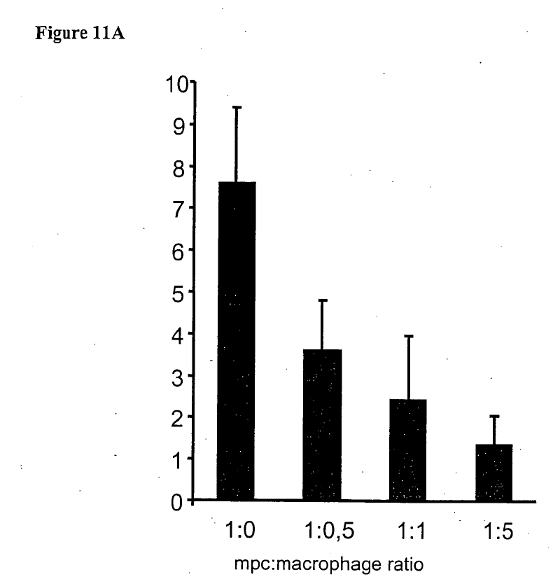

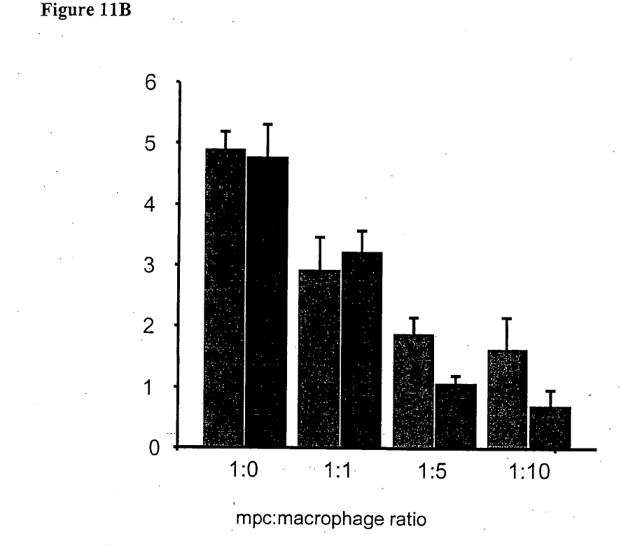

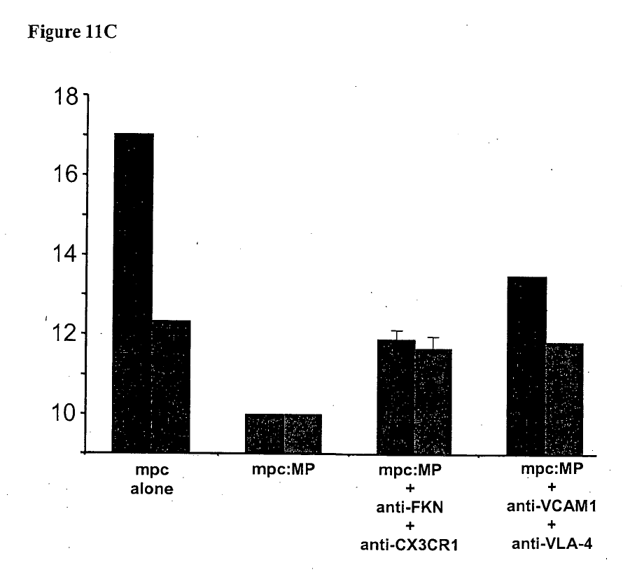

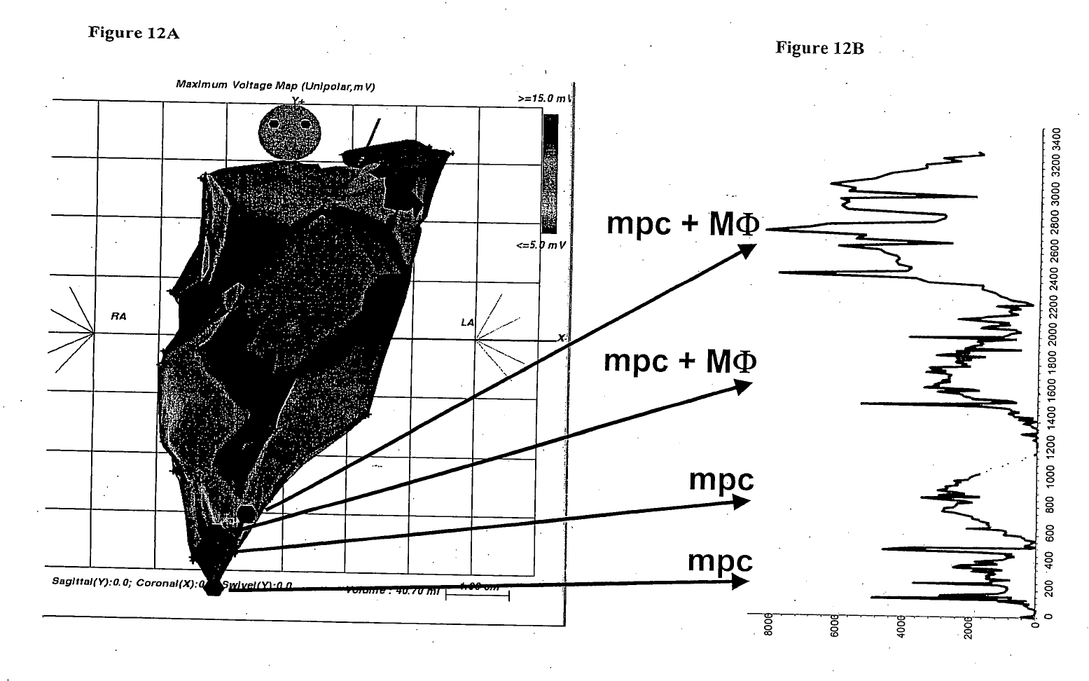

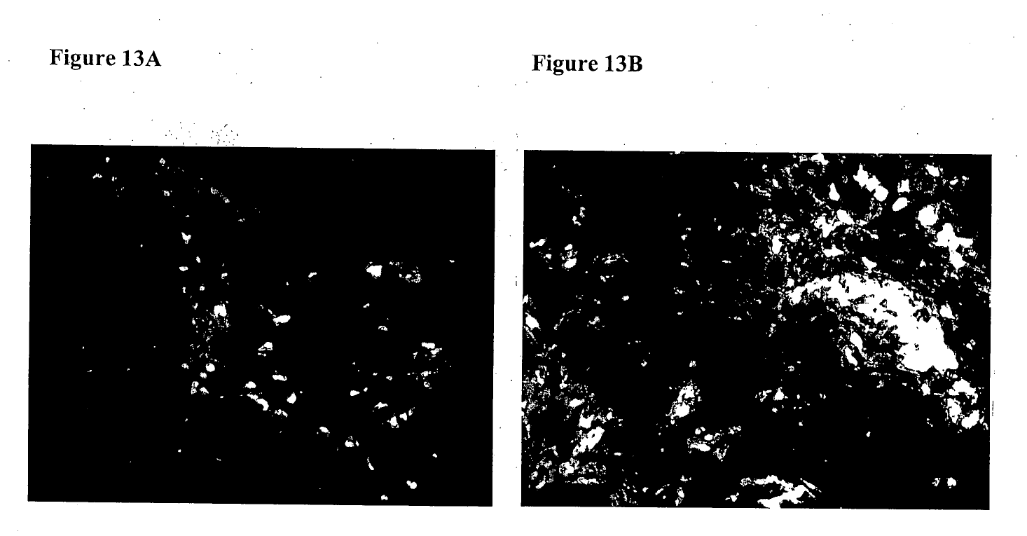

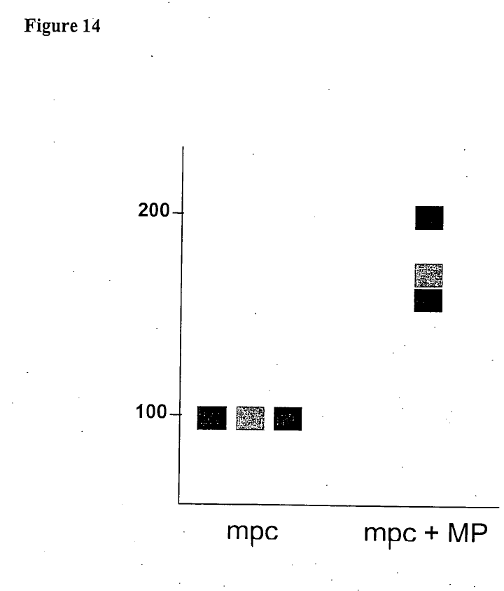

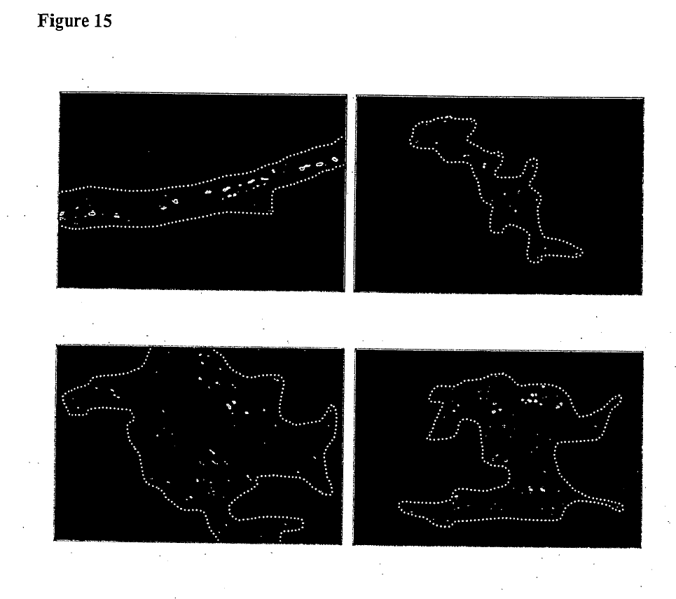

Mpc proliferation, quantified by [³H]-thymidine incorporation, was strongly promoted by MP-conditioned medium in a dose-dependent way, an increase of 126% being observed at the 1:2 (mpc:MP) ratio (p<0.004) (FIG. 9A). Mpc proliferation could be specifically evaluated in cocultures because human MP are post-mitotic cells {van der Meer, van de Gevel, et al. 1982 218/id} that do not incorporate [³H]-thymidine (FIG. 9B). Mpc proliferation was moderately decreased by direct contact with MP, a decrease of 27% being observed at the 1:2 (mpc:MP) ratio (p<0.004) (FIG. 9B). Therefore, the net cell growth increase observed in cocultures allowing cell:cell contacts could not be attributed to a mitogenic effect. The determination of oligosomal DNA levels showing much lower apoptosis in cocultures (1:1 ratio) than expected from addition of the levels determined in separated mpc and MP cultures (FIG. 10A) showed that macrophages exert an anti-apoptotic effect mediated by macrophage contacts. To discriminate between variations of apoptosis affecting mpc and MP, a double labeling with anti-CD56 antibody, a mpc marker, and annexin-V, an early marker of apoptosis, was performed. As compared with separate cultures, cocultures at 1:1 ratio showed a decreased number of both apoptotic mpc (annexin-V⁺, CD56+ cells) (48.1 vs. 17.3%, p<0.02) and apoptotic MP (annexin-V⁺, CD56⁻ cells) (63.1 vs. 39.9%, p<0.01) (FIG. 10B,C,D). Rescuing of mpc from death could not be attributed to soluble factors since mpc apoptosis remained unchanged upon administration of MP-conditioned medium (FIG. 10A).

### Example 6

### Use of Macrophages as Adjuvant of Intramyocardic Cell Therapy in Pigs

The aim of the study was the transplantation of skeletal myogenic precursor cells (mpc), alone or co-transplanted with macrophages, in pigs. Closed-chest mpc transplantation was assessed using the NOGA-Biosense® device allowing both electromechanical mapping of the left ventricle (LV), and guided mpc injections through endocardium.

A Material and Methods

Skeletal mpc were obtained from sternocleidomastoid muscles of the pigs, which were mechanically minced and incubated in digestion medium (HAM F12-HEPES containing 1,5 mg/ml pronase E (Sigma, St Louis, Mo., USA) and 0.03% EDTA (p:v)) (Invitrogen, Paisley, Scotland, UK) for 40 min at 37° C. Cells were recovered from tissue debris after washes, slow centrifugations and filtering. Cells were seeded in HAM-F12 containing 15% fetal calf serum (FCS) (Invitrogen). Cell expansion was enhanced by addition of human bFGF (10 ng/ml) and IGF-1 (50 ng/ml) (Abcys, Paris, France). Culture in the Cell Factory™ device (Nunc, Roskilde, Denmark) allowed the production of about 10⁹ cells in 4 weeks. Mpc were labelled with a fluorescent dye (PKH26, Sigma) before being transplanted alone or with macrophages

Macrophages are prepared from blood monocytes differentiated in Teflon bags containing serum (10%) in culture medium (RPMI) for 1 week, as described in previous example.

Two ratios of mpc/macrophage were used, expressed in average cell number: 1:1 and 3:1.

Transplantation into the myocardium of the cells is realized in pigs using a non surgical procedure: the NOGA-STAR™ mapping catheter from Biosense Webster, Johnson and Johnson (NOGA-BIOSENSE®). This system combines electromechanical mapping of the left ventricle, through sensing areas devoid of contractility and electric activity, and possibility of multiple guided mpc microinjections through endocardium into the target area, using a procedure sensing mural contact, i.e. appropriate to injection in a contracting heart. This endocavitary device, which is introduced into the left ventricle through a peripheral artery, avoids sternotomy and therefore has the potential interest to reduce markedly the morbidity associated with surgical mpc engraftment. Cells are injected in RPMI at 120-150×10⁶ cells/ml. One injection has a volume of 0.4 ml. About 10 to 20 injections are made in allover the infarcted area.

B. Results

It has been shown that: 1) improved preimplantation handling of mpc can be achieved when mpc are kept in 0.1% serum albumin-containing medium until implantation; 2) mpc are neither retained nor destroyed into the catheter and their passage does not affect their survival, growth and differentiation; 3) large numbers of mpc can be actually transplanted in the LV myocardium by transendocardial route, as assessed by post-mortem examination of pigs injected with iron-loaded mpc; 4) cell injection into the myocardium does not induce conspicuous cell mortality since more than 80% of mpc recovered from LV tissue are alive 15 min after injection; 5) mpc injections can be guided into circumscribed LV targets such as infarcted areas, as assessed by comparison of map injection sites with location of iron-loaded mpc at post-mortem examination of LV myocardium. This study shows that the endoventricular route allows the targeted transplantation of the cells and that the cell mortality is not related to a mechanical cause.

Comparative histological results show that the fluorescent signal, corresponding to the number of mpc, is higher when macrophages are present. The histological sections are used to quantify the fluorescent signal by using a microplate fluorescence reader.

A more detailed observation shows that, when compared to results obtained when mpcs are transplanted alone, mpcs co-transplanted with macrophages induce an increased number of myotubes, mpc's phenotype at least partly differentiated through cardiomyocyte phenotype, and connections and contacts between myotubes are observed, correlating with a better contractility of myotubes.

### Example 7

### Use of Macrophages as Adjuvant of Intramyocardic Injection of Transfected Mpcs in Pigs

Macrophages and mpc are prepared as previously described. Mpc are transfected by a lentivirus contaning lacZ (Invitrogen). Macrophages and transfected mpc, or transfected mpc alone, are injected in pig as previously described, at ratios from 1/1 to 1/3. The presence of beta-galactosidase activity is analyzed histologically and enzymatically, indicative of the survival and proliferation of mpc. The co-administration of macrophages enhances mpc's survival and proliferaion within myocardial tissue.

### Example 8

### Use of Macrophages as Adjuvant of Intramyocardic Cell Therapy in Humans

Macrophages are prepared from PBMCs blood monocytes, such as described in PCT/EP93/01232. Briefly, approximately 10×10⁹ mononuclear cells (PBMCs, with 25 to 40% monocytes) are collected from apheresis using a blood separator (COBE Spectra LRS Leukoreduction system, COBE BCT, Lakewood, Colo.). Harvested mononuclear cells are differentiated into macrophages by a 7 days culture under standard operating procedures using a specific designed device (MAK cell processor, Immuno-Designed Molecules, Paris). Monocytes are seeded in air permeable hydrophobic bags in supplemented Iscove Modified Dulbecco Medium, added with Granulocyte-Macrophage Colony Stimulating Factor (500 U/ml, Sandoz-Novartis, Rueil-Malmaison, France) and 2% of autologous serum. Macrophages are purified by elutriation (Beckman Avanti J20 centrifuge with a JE 5.0 rotor, Beckman Coultyer, Miami, Fla.) and resuspended into saline solution. A cell sample is taken for microscopic examination of morphology and assessment of CD14 and CD64 antigen expression by flow cytometry. Bacteriological controls are performed throughout the process and immediately before infusion of each MAK cell bag. On average one billion macrophages are recovered. Some 300 millions macrophages are added to 300 millions muscle cells in culture. The remaining macrophages are kept frozen in culture medium with 10% human serum albumin and 10% DMSO.

Skeletal muscles were prepared as described in Garaud et al, 2001.

Macrophages and mpcs are injected into myocardial tissue at doses of 10 million to 1 billion and preferably 10 injections of 0.5 ml of an isotonic solution containing 100 millions of cells per ml at a 1/1 macrophages/muscle cells ratio. Cell parity is established by addition of freshly thawed macrophages frozen after initial preparation.

A endocavity system is used for the injection. The therapeutic efficacy on the cardiac muscle is measured by technique chosen amongst: cardiac catheterism with left ventricular angiography, cardiac echography, magnetic resonance imaging, single photon cardiac tomography emission (SPECT), positon emission tomography (PET). This allows an objective evaluation of left ventricular global functions and ejection fractions as well as cardiac global function (contractility, viability, tissue perfusion). The therapeutic benefits observed include 1—improvement of symptoms of walking capacity and breathing stress, 2—reduction of hospitalization related to cardiac capacity, 3—reduced death frequency.

### Example 9

### Cell Engraftement after Dilated Cardiomyopathy (DCM)

Dilated cardiomyopathy (DCM) is characterized by dilation and impaired contraction of the left or both cardiac ventricles. This severe condition may progress to advanced heart failure, sudden death, or both. Histopathological changes typically include extensive ventricular areas of cardiomyocyte loss with fibrosis replacement. DCM frequently occurs in the course of skeletal myopathies, such as patients with Duchenne muscular dystrophy, in which it has a major impact on prognosis. Several hereditary forms of DCM can be caused by defects of the extrasarcomeric myocyte cytoskeleton, or by alterations within the dystrophin-glycoprotein complex. These mutated cytoskeletal and nuclear transporter proteins may alter force transmission or disrupt nuclear function, resulting in cell death (reviews in Franz et al, 2001; Emery, 2002). Although cardiac transplantation is of benefit to patients with advanced DCM, the growing donor heart deficiency limits this option.

Therefore grafts are attempted where multipotent adult stem cells are obtained as Lee (2000). Macrophages are obtained as described in example 8.

Patients are injected with multipotent adult stem cells and macrophages, administered directly at the site of missing cardiomyocytes. The conditions of treatment are similar of that of example 8. The fibrotic area is limited, so as subsequent congestive heart failure.

This strengthens the need for new therapeutic approaches, such as cell therapy that aims at replacing missing cardiomyocytes by contractile cells to limit the fibrotic area and Both multipotent adult stem (AS) cells and myogenic precursor cells (mpc), with a restricted potential of differentiation, have been used to repair damaged heart tissue. DCM is a focal muscle disease and, therefore, constitutes a choice candidate for substitutive cell therapy.

### REFERENCES

Allen, R. E., S. M. Sheehan, R. G. Taylor, T. L. Kendall, and G. M. Rice. 1995. Hepatocyte growth factor activates quiescent skeletal muscle satellite cells in vitro. J. Cell Physiol 165:307-312.
Andjelkovic, A. V., D. Kerkovich, and J. S. Pachter. 2000. Monocyte:astrocyte interactions regulate MCP-1 expression in both cell types. J. Leukoc. Biol. 68:545-552.
Astolfi, A., C. De Giovanni, L. Landuzzi, G. Nicoletti, C. Ricci, S. Croci, L. Scopece, P. Nanni, and P. L. Lollini. 2001. Identification of new genes related to the myogenic differentiation arrest of human rhabdomyosarcoma cells. Gene 274:139-149.
Authier, F. J., B. Chazaud, A. Plonquet, M. C. Eliezer-Vanerot, F. Poron, L. Belec, G. Barlovatz-Meimon, and R. K. Gherardi. 1999. Differential expression of the IL-1 system components during in vitro myogenesis: implication of IL-1b in induction of myogenic cell apoptosis. Cell Death Differ. 6:1012-1021.
Bausero, P., M. Ben Mahdi, J. Mazucatelli, C. Bloy, and M. Perrot-Applanat. 2000. Vascular endothelial growth factor is modulated in vascular muscle cells by estradiol, tamoxifen, and hypoxia. Am. J. Physiol Heart Circ. Physiol 279:H2033-H2042.
Bazan, J. F., K. B. Bacon, G. Hardiman, W. Wang, K. Soo, D. Rossi, D. R. Greaves, A. Zlotnik, and T. J. Schall. 1997. A new class of membrane-bound chemokine with a CX3C motif. Nature 385:640-644.
Cantini, M. and U. Carraro. 1995. Macrophage-released factor stimulates selectively myogenic cells in primary muscle culture. J. Neuropathol. Exp. Neurol. 54:121-128.
Cantini, M., M. L. Massimino, E. Rapizzi, K. Rossini, C. Catani, L. Dalla Libera, and U. Carraro. 1995. Human satellite cell proliferation in vitro is regulated by autocrine secretion of IL-6 stimulated by a soluble factor(s) released by activated monocytes. Biochem. Biophys. Res. Commun. 216:49-53.
Carpenter, S. and G. Karpati. 2001. General aspects of skeletal muscle biology. In Pathology of skeletal muscle. S. Carpenter and G. Karpati, editors. Oxford University Press, New York. 28-62.
Chapman, G. A., K. E. Moores, J. Gohil, T. A. Berkhout, L. Patel, P. Green, C. H. Macphee, and B. R. Stewart. 2000. The role of fractalkine in the recruitment of monocytes to the endothelium. Eur. J. Pharmacol. 392:189-195.
Chazaud, B., S. Bonavaud, A. Plonquet, M. Pouchelet, R. K. Gherardi, and G. Barlovatz-Meimon. 2000. Involvement of the [uPAR:uPA:PAI-1:LRP] Complex in Human Myogenic Cell Motility. Exp. Cell Res. 258:237-244.
Chazaud, B., L. Hittinger, C. Sonnet, S. Champagne, P. Le Corvoisier, N. Benhaiem-Sigaux, T. Unterseeh, P. Merlet, A. Rahmouni, J. Garot, R. K. Gherardi, and E. Teiger. 2003. Endoventricular porcine autologous myoblast transplantation can be successfully achieved with minor mechanical cell damage. Cardiovasc. Res. In Press.
Chazaud, B., R. Ricoux, C. Christov, A. Plonquet, R. K. Gherardi, and G. Barlovatz-Meimon. 2002. Promigratory effect of plasminogen activator inhibitor-1 on invasive breast cancer cell populations. Am. J. Pathol. 160:237-246.
Confalonieri, P., P. Bernasconi, P. Megna, S. Galbiati, F. Cornelio, and R. Mantegazza. 2000. Increased expression of beta-chemokines in muscle of patients with inflammatory myopathies. J. Neuropathol. Exp. Neurol. 59:164-169.
De Rossi, M., P. Bemasconi, F. Baggi, R. de Waal Malefyt, and R. Mantegazza. 2000. Cytokines and chemokines are both expressed by human myoblasts: possible relevance for the immune pathogenesis of muscle inflammation. Int. Immunol. 12:1329-1335.
Dee, K., M. Freer, Y. Mei, and C. M. Weyman. 2002. Apoptosis coincident with the differentiation of skeletal myoblasts is delayed by caspase 3 inhibition and abrogated by MEK-independent constitutive Ras signaling. Cell Death. Differ. 9:209-218.
Fantuzzi, L., P. Borghi, V. Ciolli, G. Pavlakis, F. Belardelli, and S. Gessani. 1999. Loss of CCR2 expression and functional response to monocyte chemotactic protein (MCP-1) during the differentiation of human monocytes: role of secreted MCP-1 in the regulation of the chemotactic response. Blood 94:875-883.
Felker et al, 2000. N EngL. J Med, 342: 1077.
Festoff, B. W., R. B. Reddy, M. VanBecelaere, I. Smimova, and J. Chao. 1994. Activation of serpins and their cognate proteases in muscle after crush injury. J. Cell Physiol 159:11-18.
Fong, A. M., L. A. Robinson, D. A. Steeber, T. F. Tedder, O. Yoshie, T. Imai, and D. D. Patel. 1998. Fractalkine and CX3CR1 mediate a novel mechanism of leukocyte capture, firm adhesion, and activation under physiologic flow. J. Exp. Med. 188:1413-1419.
Foussat, A., L. Bouchet-Delbos, D. Berrebi, I. Durand-Gasselin, A. Coulomb-L'Hermine, R. Krzysiek, P. Galanaud, Y. Levy, and D. Emilie. 2001. Deregulation of the expression of the fractalkine/fractalkine receptor complex in HIV-1-infected patients. Blood 98:1678-1686.
Foxman, E. F., J. J. Campbell, and E. C. Butcher. 1997. Multistep navigation and the combinatorial control of leukocyte chemotaxis. J. Cell Biol. 139:1349-1360.
Foxman, E. F., E. J. Kunkel, and E. C. Butcher. 1999. Integrating conflicting chemotactic signals: the role of memory in leukocyte navigation. J. Cell Biol. 147:577-587.
Garaud et al, 2001. Lancet. 357, 279-280.
Gordon, S. 1995. The macrophage. Bioessays 17:977-986.
Grounds, M. D. 1987. Phagocytosis of necrotic muscle in muscle isografts is influenced by the strain, age, and sex of host mice. J. Pathol. 153:71-82.
Guerin, C. W. and P. C. Holland. 1995. Synthesis and secretion of matrix-degrading metalloproteases by human skeletal muscle satellite cells. Dev. Dyn. 202:91-99.
Hawke, T. J. and D. J. Garry. 2001. Myogenic satellite cells: physiology to molecular biology. J. Appl. Physiol. 91:534-551.
Imaizumi, T., T. Matsumiya, K. Fujimoto, K. Okamoto, X. Cui, U. Ohtaki, Hidemi, Yoshida, and K. Satoh. 2000. Interferon-gamma stimulates the expression of CX3CL1/fractalkine in cultured human endothelial cells. Tohoku J. Exp. Med. 192:127-139.
Jesse, T. L., R. LaChance, M. F. Iademarco, and D. C. Dean. 1998. Interferon regulatory factor-2 is a transcriptional activator in muscle where It regulates expression of vascular cell adhesion molecule-1. J. Cell Biol. 140:1265-1276.
Katou, F., H. Ohtani, T. Nakayama, K. Ono, K. Matsushima, A. Saaristo, H. Nagura, O. Yoshie, and K. Motegi. 2001. Macrophage-derived chemokine (MDC/CCL22) and CCR4 are involved in the formation of T lymphocyte-dendritic cell clusters in human inflamed skin and secondary lymphoid tissue. Am. J. Pathol. 158:1263-1270.
Katschke, K. J., Jr., J. B. Rottman, J. H. Ruth, S. Qin, L. Wu, G. LaRosa, P. Ponath, C. C. Park, R. M. Pope, and A. E. Koch. 2001. Differential expression of chemokine receptors on peripheral blood, synovial fluid, and synovial tissue monocytes/macrophages in rheumatoid arthritis. Arthritis Rheum. 44:1022-1032.
Lee, J Y, Qu-Petersen, Z, Cao, B, Kimura, S, Jankowski, R, Cummins, J, Usas, A, Gates, C, Robbins, P, Wernig, A, and Huard, J. 2000. Clonal isolation of muscle-derived cells capable of enhancing muscle regeneration and bone healing. J Cell Biol. 150:1085-1100.
Lescaudron, L., E. Peltekian, J. Fontaine-Pérus, D. Paulin, M. Zampieri, L. Garcia, and E. Parrish. 1999. Blood borne macrophages are essential for the triggering of muscle regeneration following muscle transplant. Neuromuscul. Disord. 9:72-80.
Liprandi, A., C. Bartoli, D. Figarella-Branger, J. F. Pellissier, and H. Lepidi. 1999. Local expression of monocyte chemoattractant protein-1 (MCP-1) in idiopathic inflammatory myopathies. Acta Neuropathol. (Berl) 97:642-648.
Lluis, F., J. Roma, M. Suelves, M. Parra, G. Aniorte, E. Gallardo, I. Illa, L. Rodriguez, S. M. Hughes, P. Carmeliet, M. Roig, and P. Munoz-Canoves. 2001. Urokinase-dependent plasminogen activation is required for efficient skeletal muscle regeneration in vivo. Blood 97:1703-1711.
Lucas, A. D., N. Chadwick, B. F. Warren, D. P. Jewell, S. Gordon, F. Powrie, and D. R. Greaves. 2001. The transmembrane form of the CX3CL1 chemokine fractalkine is expressed predominantly by epithelial cells in vivo. Am. J. Pathol. 158:855-866.
Mantovani, A., P. A. Gray, J. Van Damme, and S. Sozzani. 2000. Macrophage-derived chemokine (MDC). J. Leukoc. Biol. 68:400-404.
Matsukawa, A., C. M. Hogaboam, N. W. Lukacs, P. M. Lincoln, H. L. Evanoff, and S. L. Kunkel. 2000. Pivotal role of the CC chemokine, macrophage-derived chemokine, in the innate immune response. J. Immunol. 164:5362-5368.
McLennan, I. S. 1996. Degenerating and regenerating skeletal muscles contain several subpopulations of macrophages with distinct spatial and temporal distributions. J. Anat. 188:17-28.
McQuibban, G. A., J. H. Gong, J. P. Wong, J. L. Wallace, I. Clark-Lewis, and C. M. Overall. 2002. Matrix metalloproteinase processing of monocyte chemoattractant proteins generates CC chemokine receptor antagonists with anti-inflammatory properties in vivo. Blood 100:1160-1167.
Merly, F., L. Lescaudron, T. Rouaud, F. Crossin, and M.-F. Gardahaut. 1999. Macrophages enhance muscle satellite cell proliferation and delay their differentiation. Muscle Nerve 22:724-732.
Meszaros et al. 1999. Macrophages phagocytosis of wound neutrophils. J. Leukocyte Biology, Vol. 65, p. 35.
Meucci, O., A. Fatatis, A. A. Simen, and R. J. Miller. 2000. Expression of CX3CR1 chemokine receptors on neurons and their role in neuronal survival. Proc. Natl. Acad. Sci. U.S. A 97:8075-8080.
Parrish, E. P., C. Cifuentes-Diaz, Z. L. Li, P. Vicart, D. Paulin, P. A. Dreyfus, M. Peschanski, A. J. Harris, and L. Garcia. 1996. Targeting widespread sites of damage in dystrophic muscle: engrafted macrophages as potential shuttles. Gene Ther. 3:13-20.
Pimorady-Esfahani, A., M. D. Grounds, and P. G. McMenamin. 1997. Macrophages and dendritic cells in normal and regenerating murine skeletal muscle. Muscle Nerve 20:158-166.
Polazzi, E., T. Gianni, and A. Contestabile. 2001. Microglial cells protect cerebellar granule neurons from apoptosis: evidence for reciprocal signaling. Glia 36:271-280.
Preissner, K. T., S. M. Kanse, and A. E. May. 2000. Urokinase receptor: a molecular organizer in cellular communication. Curr. Opin. Cell Biol 12:621-628.
Quax, P. H. A., E. Frisdal, N. Pedersen, S. Bonavaud, P. Thibert, I. Martelly, J. H. Verheijen, F. Blasi, and G. Barlovatz-Meimon. 1992. Modulation of activities and RNA level of the components of the plasminogen activation system during fusion of human myogenic satellite cells in vitro. Dev. Biol. 151:166-175.
Rapalino, O., O. Lazarov-Spiegler, E. Agranov, G. J. Velan, E. Yoles, M. Fraidakis, A. Solomon, R. Gepstein, A. Katz, M. Belkin, M. Hadani, and M. Schwartz. 1998. Implantation of stimulated homologous macrophages results in partial recovery of paraplegic rats. Nat. Med. 4:814-821.
Resnati, M., M. Guttinger, S. Valcamonica, N. Sidenius, F. Blasi, and F. Fazioli. 1996. Proteolytic cleavage of the urokinase receptor substitutes for the agonist-induced chemotactic effect. EMBO J. 15:1572-1582.
Reyes-Reyna, S. M. and K. A. Krolick. 2000. Chemokine production by rat myocytes exposed to interferon-gamma. Clin. Immunol. 94:105-113.
Rissanen, T. T., I. Vajanto, M. O. Hiltunen, J. Rutanen, M. I. Kettunen, M. Niemi, P. Leppanen, M. P. Turunen, J. E. Markkanen, K. Arve, E. Alhava, R. A. Kauppinen, and S. Yla-Herttuala. 2002. Expression of vascular endothelial growth factor and vascular endothelial growth factor receptor-2 (KDR/Flk-1) in ischemic skeletal muscle and its regeneration. Am. J. Pathol. 160:1393-1403.
Robertson, T. A., M. A. L. Maley, M. D. Grounds, and J. M. Papadimitriou. 1993. The role of macrophages in skeletal muscle regeneration with particular reference to chemotaxis. Exp. Cell Res. 207:321-331.
Rosen, G. D., J. R. Sanes, R. LaChance, J. M. Cunningham, J. oman, and D. C. ean. 1992. Roles for the integrin VLA-4 and its counter receptor VCAM-1 in myogenesis. Cell 69:1107-1119.
Ruth, J. H., M. V. Volin, G. K. Haines, III, D. C. Woodruff, K. J. Katschke, Jr., J. M. Woods, C. C. Park, J. C. Morel, and A. E. Koch. 2001. Fractalkine, a novel chemokine in rheumatoid arthritis and in rat adjuvant-induced arthritis. Arthritis Rheum. 44:1568-1581.
Sadahira, Y. and M. Mori. 1999. Role of the macrophage in erythropoiesis. Pathol. Int. 49:841-848.
Saghizadeh, M., J. M. Ong, W. T. Garvey, R. R. Henry, and P. A. Kern. 1996. The expression of TNF alpha by human muscle. Relationship to insulin resistance. J. Clin. Invest 97:1111-1116.
Sata et al. 2002. Hematopoietic stem cells differentiate into vascular cells that participate in the pathogenesis of atherosclerosis. Nature Medicine. Vol. 8, N° 4, April 2002.
Sawano, A., S. Iwai, Y. Sakurai, M. Ito, K. Shitara, T. Nakahata, and M. Shibuya. 2001. Flt-1, vascular endothelial growth factor receptor 1, is a novel cell surface marker for the lineage of monocyte-macrophages in humans. Blood 97:785-791.
Schmalbruch, H. and U. Hellhammer. 1977. The number of nuclei in adult rat muscles with special reference to satellite cells. Anat. Rec. 189:169-175.
Seale, P., A. Asakura, and M. A. Rudnicki. 2001. The potential of muscle stem cells. Dev. Cell 1:333-342.
Sidenius, N., C. F. Sier, and F. Blasi. 2000. Shedding and cleavage of the urokinase receptor (uPAR): identification and characterisation of uPAR fragments in vitro and in vivo. FEBS Lett. 475:52-56.
Skuk, D. and J. P. Tremblay. 2000. Progress in myoblast transplantation: a potential treatment of dystrophies. Microsc. Res. Tech. 48:213-222.
Spradling, A., D. Drummond-Barbosa, and T. Kai. 2001. Stem cells find their niche. Nature 414:98-104.
Takeishi, T., K. Hirano, T. Kobayashi, G. Hasegawa, K. Hatakeyama, and M. Naito. 1999. The role of Kupffer cells in liver regeneration. Arch. Histol. Cytol. 62:413-422.
Tidball, J. G., E. Berchenko, and J. Frenette. 1999. Macrophage invasion does not contribute to muscle membrane injury during inflammation. J. Leukoc. Biol. 65:492-498.
Vachon P H, F. A. U., H. F. Xu, L. F. Liu, F. F. Loechel, Y. F. Hayashi, K. F. Arahata, F. A. U. Reed J C, F. A. U. Wewer U M, and E. Engvall. 1997. Integrins (alpha7beta1) in muscle function and survival. Disrupted expression in merosin-deficient congenital muscular dystrophy. J. Clin. Invest 100:1870-1881.
van der Meer, J. W., J. S. van de Gevel, A. A. Blusse van Oud, J. A. Kramps, T. L. van Zwet, P. C. Leijh, and R. van Furth. 1982. Characteristics of human monocytes cultured in the Teflon culture bag. Immunology 47:617-625.
Volin, M. V., J. M. Woods, M. A. Amin, M. A. Connors, L. A. Harlow, and A. E. Koch. 2001. Fractalkine: a novel angiogenic chemokine in rheumatoid arthritis. Am. J. Pathol. 159:1521-1530.
Zachariae, C. O. C., C. G. Larsen, and K. Matsushima. 1998. Monocyte chemoattractant protein 1. In Encyclopedia of Immunology. P. J. Delves and I. M. Roitt, editors. Academic Press, London. 1748-1450.
Zhao et al. 2003. A human peripheral blood monocyte-derived subset acts as pluripotent stem cells. PNAS, Vol. 100, N° 5, March 2003.

1-70. (canceled)

71. A method for the treatment of a disease or of a lesion involving cellular apoptosis, reduction of the survival of cells and/or destruction of cells, comprising the administering of an appropriate amount of:
macrophages or
macrophage-conditioned medium or
macrophages and macrophage-conditioned medium.

72. The method according to claim 71 for the improvement of survival of a first type of cells, for the treatment of a disease or of a lesion involving the destruction of a second type of cells or of a tissue containing said second type of cells, said first type of cells being chosen among the group consisting of precursor cells and stem cells, said second type of cells being chosen among the group consisting of precursor cells, stem cells and any type of differentiated cells, comprising treating said cells with macrophages.

73. The method according to claim 71 for the improvement of survival of a first type of cells, for the treatment of a disease or of a lesion involving the destruction of a second type of cells or of a tissue containing said second type of cells, said first type of cells being chosen among the group consisting of precursor cells and stem cells, said second type of cells being chosen among the group consisting of precursor cells, stem cells and any type of differentiated cells, comprising culturing said cells in a macrophage-conditioned medium.

74. The method according to claim 72, for the treatment of one or several focal lesions wherein said first type of cells is to be grafted into a mammal.

75. The method according to claim 73, for the treatment of one or several focal lesions wherein said first type of cells is to be grafted into a mammal.

76. The method according to claim 72, wherein said first type of cells and/or macrophages are autologous for said mammal.

77. The method according to claim 73, wherein said first type of cells and/or macrophages are autologous for said mammal.

78. The method according to claim 71, for the treatment of bone or of muscular lesion, or for the treatment of cardiac lesion, said cardiac lesion being possibly myocardial infarction, coronary thrombosis, dilated cardiomyopathy or cardiomyocyte dysfunction subsequent to, or resulting from, a genetic defect.

79. The method according to claim 72, wherein macrophages act as a inhibitors of apoptosis of said first type of cells by cell to cell contact between the surface of respectively said macrophages and said first type of cells.

80. The method according to claim 73, wherein said first type of cells and/or macrophages are autologous for said mammal.

81. The method according to claim 72, wherein macrophages act as a stromal support for said first type of cells.

82. The method according to claim 73, wherein macrophages act as a stromal support for said first type of cells.

83. The method according to claim 72, wherein said first type of cells is chosen among a group consisting of: myogenic precursor cells, endothelial precursor cells, hematopoietic precursor cells, bone marrow precursor cells, mesenchymal precursor cells, neuronal precursor cells and multipotent adult stem cells.

84. The method according to claim 73, wherein said first type of cells is chosen among a group consisting of: myogenic precursor cells, endothelial precursor cells, hematopoietic precursor cells, bone marrow precursor cells, mesenchymal precursor cells, neuronal precursor cells and multipotent adult stem cells.

85. A method of preparation of a composition to be grafted into a mammal, comprising administering a composition containing:
macrophages or
macrophage-conditioned medium or
macrophages and macrophage-conditioned medium,
and at least one first type of cells,
in association with a pharmaceutically acceptable vehicle,
said first type of cells being chosen among the group consisting of: precursor cells and stem cells.

86. The method of preparation of a composition to be grafted into a mammal according to claim 85, wherein said first type of cells are autologous to said mammal or wherein said first type of cells are myogenic precursor cells.

87. The method of preparation of a composition to be grafted into a mammal according to claim 85, for the treatment of a disease or of a lesion involving the destruction of cells, or for the treatment of one or several focal lesions or for the treatment of bone or muscular lesion, or for the treatment of cardiac lesion, said cardiac lesion being possibly myocardial infarction, coronary thrombosis, dilated cardiomyopathy or cardiomyocyte dysfunction resulting from a genetic defect.

88. The method of preparation of a composition to be grafted into a mammal according to claim 85, wherein said composition contains from about 0.5 10⁸ to about 7.5 108 macrophages and from about 0.5 10⁸ to about 7.5 10⁸ of said first type of cells.

89. A pharmaceutical composition containing at least one first type of cells, said first type of cells being possibly
precursor cells or stem cells, and macrophages, or
precursor cells or stem cells, and macrophage-conditioned medium,
in association with a pharmaceutically acceptable vehicle.

90. The pharmaceutical composition according to claim 89, wherein said first type of cells is chosen among a group consisting of: myogenic precursor cells, endothelial precursor cells, hematopoietic precursor cells, bone marrow precursor cells, mesenchymal precursor cells, neuronal precursor cells and multipotent adult stem cells.

91. The pharmaceutical composition according to claim 89, wherein the ratio between said first type of cells and macrophages, as expressed in number of cells, is comprised between about 1/10 and about 10/1, and is preferably of about 1/1.

92. The pharmaceutical composition according to claim 89, wherein the percentage of macrophages, expressed in relation to the total number of cells in the composition, is from about 5% to about 70%, and more preferably from about 20% to about 50%, and more preferably of about 35%.

93. The pharmaceutical composition according to claim 89, containing frozen precursors cells or stem cells on one hand and frozen macrophages on other hand, in pharmaceutically acceptable cryopreservant and vehicle.

94. The pharmaceutical composition according to claim 89, containing macrophages and myogenic precursor cells.

95. The pharmaceutical composition according to claim 89, containing macrophage-conditioned medium and myogenic precursor cells.

96. The pharmaceutical composition according to claim 89, containing macrophages and myogenic precursor cells or containing macrophage-conditioned medium and myogenic precursor cells, wherein the ratio between macrophages and myogenic precursor cells, as expressed in number of cells, is comprised between about 1/10 and about 10/1, and preferably of about 1/1.

97. The pharmaceutical composition according to claim 89, containing macrophages and myogenic precursor cells or containing macrophage-conditioned medium and myogenic precursor cells, wherein the percentage of cells, expressed in relation of the total number of cells in the composition, is comprised from about 10% to about 80% of macrophages, more preferably about 50%, and from about 10% to about 80% of myogenic cell precursor cells, more preferably about 50%.

98. The pharmaceutical composition according to claim 89, containing from about 0.5 10⁸ to about 7.5 10⁸ and preferably from about 1.5 10⁸ to about 2.5 10⁸ macrophages.

99. The pharmaceutical composition according to claim 89, containing macrophages and myogenic precursor cells or containing macrophage-conditioned medium and myogenic precursor cells, containing from about 0.5 10⁸ to about 7.5 10⁸ and preferably from about 1.5 10⁸ to about 2.5 10⁸ myogenic precursor cells.

100. A binary complex made of a myogenic precursor cell and a macrophage, interacting by cell to cell contacts between surface receptors on the surface of, respectively, macrophage and myogenic precursor cell.

101. The binary complex according to claim 100, wherein cell to cell contacts are mediated, at least partly, via cell surface molecules VLA4 and VCAM1 or via cell surface molecules fractalkine (CX3CL1) and CX3CR1, on the surface of myogenic precursor cell and macrophage.

102. Process for preparing pharmaceutical compositions according to claim 89, containing:
a first type of cells
and macrophages or macrophage-conditioned medium,
said process comprising contacting
a first type of cells, chosen among the group consisting of precursor cells and stem cells,
and macrophages or macrophage-conditioned medium.

103. The process for preparing pharmaceutical compositions according to claim 102 wherein said first type of cells and said macrophages or macrophage-conditioned medium are contacted for a time sufficient to allow at least one cycle of cellular division of said first type of cells.

104. A product containing macrophages or a macrophage-conditioned medium and a first type of cells, being possibly precursor cells or stem cells, as a combined preparation for the separate, simultaneous or sequential administration of a cellular graft into a mammal.

105. The product according to claim 104, wherein precursor cells are myogenic precursor cells.

106. The product according to claim 104, where aliquots of the first type of cells and the macrophages are kept frozen in acceptable vehicle until thawing for the injection.
